(19) United States
(12) Patent Application Publication
(10) Pub. No.: US 2025/0176958 A1
(43) Pub. Date: Jun. 5, 2025

(54) END TOOL AND SURGICAL INSTRUMENT

(71) Applicant: LIVSMED INC., Seongnam-si (KR)

(72) Inventor: Woo Jung CHOI, Siheung-si (KR)

(21) Appl. No.: 18/955,807

(22) Filed: Nov. 21, 2024

(30) Foreign Application Priority Data
Dec. 1, 2023 (KR) 10-2023-0172508

(51) Int. Cl. A61B 17/04

(52) U.S. Cl. CPC A61B 17/0469

(57) ABSTRACT

An end tool includes a jaw configured to accommodate at least one region of an operation member that is movable in at least one direction, a reciprocating moving part spaced apart from the operation member, disposed on a proximal end side of the jaw rather than the operation member, and configured to receive a driving force from a separate driving part and perform at least a linear reciprocating motion, and a plurality of transmission gears configured to receive a driving force of a linear motion of the reciprocating moving part, perform a rotational motion, and transmit the driving force to the operation member so that the operation member is moved in the one direction.

### CROSS-REFERENCE TO RELATED APPLICATION

This application is based on and claims priority under 35 USC § 119 to Korean Patent Application No. 10-2023-0172508, filed on Dec. 1, 2023, in the Korean Intellectual Property Office, the disclosure of which is incorporated by reference herein in its entirety.

### BACKGROUND

### 1. Field

The disclosure relates to an end tool and a surgical instrument.

### 2. Description of the Related Art

Recently, laparoscopic surgery, which is capable of reducing postoperative recovery time and complications through small incisions, has been actively used. Laparoscopic surgery is a method of performing surgery by drilling a plurality of small holes in a patient's abdomen and observing an abdominal cavity through the small holes. Laparoscopic surgery is widely used in general surgery.

In order to perform such laparoscopic surgery, various instruments are used. For example, a suture device that is inserted into a body is used to suture a surgical site within an abdominal cavity. A surgical stapler that sutures a surgical site by using medical staples is used as the suture device.

In general, a surgical stapler is a medical device that is often used for cutting and anastomosis of organs in abdominal and thoracic organ surgery. Such surgical staplers include an open stapler used in a thoracotomy or laparotomy state and an endo stapler used in thoracoscopy and laparoscopy.

Since surgical staplers are capable of simultaneously cutting a surgical site and performing anastomosis of organs, the surgical time is reduced and the surgical site is accurately sutured. In addition, surgical staplers are widely used in modern surgical procedures due to advantages of faster recovery and less scarring than when using surgical sutures for tissue cutting and suturing. In particular, surgical staplers are widely used in cancer surgery to cut cancer tissue and suture the cut site.

### SUMMARY

The disclosure provides an end tool and a surgical instrument, in which an operation member is precisely moved when performing one or more operations included in laparoscopic surgery or various other surgeries.

Additional aspects will be set forth in part in the description which follows and, in part, will be apparent from the description, or may be learned by practice of the presented embodiments of the disclosure.

According to an embodiment, an end tool includes a jaw configured to accommodate at least one region of an operation member that is movable in at least one direction, a reciprocating moving part spaced apart from the operation member, disposed on a proximal end side of the jaw rather than the operation member, and configured to receive a driving force from a separate driving part and perform at least a linear reciprocating motion, and a plurality of transmission gears configured to receive a driving force of a linear motion of the reciprocating moving part, perform a rotational motion, and transmit the driving force to the operation member so that the operation member is moved in the one direction.

In the present embodiment, the end tool may further include at least one driving gear including a plurality of teeth and configured to come into contact with a region of the reciprocating moving part, perform a rotational motion by the reciprocating moving part, and transmit the rotational motion to one transmission gear among the plurality of transmission gears.

In the present embodiment, the reciprocating moving part may include a main body and at least one contact part extending from the main body in a direction toward the operation member, and the at least one contact part may be configured to come into contact with the at least one driving gear and transmit a force so that the driving gear is rotated.

In the present embodiment, when the at least one driving gear is rotated, a rotational force may be transmitted to the one transmission gear connected to the at least one driving gear and the plurality of transmission gears sequentially disposed, so that the plurality of transmission gears are rotated.

In the present embodiment, the at least one contact part of the reciprocating moving part may include a first contact part and a second contact part that extend from the main body, are spaced apart from each other, and have different lengths.

In the present embodiment, the at least one driving gear may include a first driving gear and a second driving gear respectively corresponding to the first contact part and the second contact part.

In the present embodiment, the first driving gear may have the form of a ratchet that comes into contact with the first contact part and is rotated in one rotation direction by the first contact part, and the second driving gear may have the form of a ratchet that comes into contact with the second contact part and is rotated in a rotation direction opposite to the one rotation direction by the second contact part.

In the present embodiment, the operation member may be moved in one straight direction, either forward or backward, through rotation of the first driving gear in the one rotation direction, and the operation member may be moved in a direction opposite to the one straight direction through rotation of the second driving gear in the rotation direction opposite to the one rotation direction.

In the present embodiment, the first driving gear and the second driving gear may be configured to be rotated around a same rotation shaft.

In the present embodiment, the first driving gear and the second driving gear may be disposed in a stacked form.

In the present embodiment, the first contact part and the second contact part may include protrusion parts formed to have different relative heights so as to respectively correspond to and come into contact with the first driving gear and the second driving gear.

In the present embodiment, a rotational force through rotation of the second driving gear may be transmitted to the plurality of transmission gears through the first driving gear.

In the present embodiment, a rack member may be disposed in at least one region of the jaw, the rack member may be configured to engage with the plurality of transmission gears, the rack member may be configured to be moved forward and backward by a driving force of the plurality of transmission gears, and the operation member may be configured to be moved by the rack member.

In the present embodiment, the operation member may be configured to be moved integrally with the rack member.

In the present embodiment, the operation member may include a main body, a blade, and a side portion connected to the main body and formed on a side of the main body opposite to a side facing the blade, the rack member may be disposed between a first side portion and a second side portion of the side portion, which are spaced apart from each other, and the rack member may include a first rack member corresponding to the first side portion and a second rack member corresponding to the second side portion.

In the present embodiment, the plurality of transmission gears may include a plurality of first transmission gears and a plurality of second transmission gears disposed to be sequentially misaligned with each other, the plurality of first transmission gears may be engaged with the first rack member, and the plurality of second transmission gears may be engaged with the second rack member.

In the present embodiment, the jaw may include a first jaw and a second jaw facing each other and configured to perform an open motion and a close motion, the first jaw may include an accommodation part configured to accommodate a plurality of staples, and the rack member may be disposed in a region opposite to a surface of the first jaw on which the accommodation part is formed.

In the present embodiment, the plurality of transmission gears may be disposed in the region opposite to the surface of the first jaw on which the accommodation part is formed, so as to correspond to the rack member.

In the present embodiment, the first jaw may include an extension groove formed along a longitudinal direction of the first jaw on a surface opposite to the surface of the first jaw on which the accommodation part is formed, and the rack member and the plurality of transmission gears may be disposed in the extension groove.

In the present embodiment, the rack member may be formed separately from the operation member, may be disposed closer to a proximal end of the jaw than the operation member, and may push the operation member during the motion of the rack member so that the operation member is moved.

In the present embodiment, the end tool may further include a driving gear including a concavo-convex portion having a plurality of teeth on an outer circumferential surface thereof and a non-formation area which is adjacent to the concavo-convex portion and in which teeth are not formed, the driving gear being in contact with one region of the reciprocating moving part and being configured to perform a rotation motion by the reciprocating moving part and transmit the rotation motion to one transmission gear among the plurality of transmission gears.

In the present embodiment, the reciprocating moving part may include a first rack gear part and a second rack gear part facing each other, and a main body connecting the first rack gear part to the second rack gear part, and the first rack gear part and the second rack gear part may be formed to have concavo-convex portions corresponding to the plurality of teeth of the driving gear.

In the present embodiment, when the reciprocating moving part is moved forward or backward, the first rack gear part or the second rack gear part may be engaged with the plurality of teeth of the driving gear so that the driving gear is rotated, and a rotational force of the driving gear may be transmitted to one transmission gear connected to the driving gear among the plurality of transmission gears and the plurality of transmission gears sequentially disposed, so that the plurality of transmission gears are rotated.

In the present embodiment, when the reciprocating moving part starts a forward motion and repeats forward and backward motions in a state in which the second rack gear part of the reciprocating moving part is engaged with the plurality of teeth of the driving gear, the driving gear may be rotated in one rotation direction and the operation member may be moved forward.

In the present embodiment, when the reciprocating moving part starts a forward motion and repeats forward and backward motions in a state in which the first rack gear part of the reciprocating moving part is engaged with the plurality of teeth of the driving gear, the driving gear may be rotated in one rotation direction and the operation member may be moved backward.

In the present embodiment, the reciprocating moving part may include a first reciprocating moving part and a second reciprocating moving part that are disposed up and down and moved forward or backward.

In the present embodiment, the first reciprocating moving part and the second reciprocating moving part may have different offsets in a direction facing the operation member and may be disposed to be moved forward or backward so as to be misaligned with each other.

In the present embodiment, the driving gear may include a first driving gear corresponding to the first reciprocating moving part and a second driving gear corresponding to the second reciprocating moving part. In the present embodiment, the first driving gear and the second driving gear may be disposed in a stacked form and may be rotated around a same rotation shaft in a same direction.

In the present embodiment, a time point at which the non-formation area of the first driving gear corresponds to the concavo-convex portion of the first driving gear and a time point at which the non-formation area of the second driving gear corresponds to the concavo-convex portion of the second driving gear may be formed to be at least partially different from each other.

In the present embodiment, the non-formation area of the first driving gear and the non-formation area of the second driving gear may be formed not to be in parallel to each other.

According to another embodiment, an end tool includes a jaw configured to accommodate at least one region of an operation member that is movable in at least one direction, a belt part connected to the at least one region of the operation member and configured to perform a loop rotational motion, and a driving module configured to support the belt part and transmit a driving force to the belt part so that the belt part is rotated.

In the present embodiment, the operation member may include a main body, and a first side portion and a second side portion connected to the main body and spaced apart from each other, the belt part may be disposed in a space between the first side portion and the second side, a region of the belt part facing the first side portion may be connected and coupled to the first side portion and moved together, and a region of the belt part facing the second side portion may be spaced apart from the second side portion.

In the present embodiment, the driving module may include a first driving pulley and a second driving pulley disposed to be spaced apart from each other, and the belt part may be configured to perform a loop motion while being wound around the first driving pulley and the second driving pulley.

In the present embodiment, the belt part may be rotated in one direction and a direction opposite thereto by using the first driving pulley and the second driving pulley.

In the present embodiment, when the belt part is rotated in the one direction and the direction opposite thereto, the operation member may be configured to be moved forward and backward between the first driving pulley and the second driving pulley.

In the present embodiment, the jaw may include a first jaw and a second jaw facing each other and configured to perform an open motion and a close motion, the first jaw may include an accommodation part configured to accommodate a plurality of staples, and the belt part may be disposed in a region of the first jaw opposite to a surface on which the accommodation part is formed.

According to another embodiment, an end tool includes a jaw configured to accommodate at least one region of an operation member that is movable in at least one direction, a motion wire connected to the at least one region of the operation member and configured to perform at least a forward motion and a backward motion, and a driving unit configured to support the motion wire and perform a rotational motion so that a driving force is transmitted to the motion wire.

In the present embodiment, the driving unit may include a driving fixing unit and a support unit disposed to be spaced apart from each other, one end region of the motion wire may be fixed to the driving fixing unit, the motion wire may be wound around the support unit to guide a path so that another end region of the motion wire is connected to the operation member, the motion wire may be wound by a rotational motion of the driving fixing unit, and the operation member may be moved forward.

In the present embodiment, the driving fixing unit may be configured to wind the motion wire multiple times.

In the present embodiment, the driving fixing unit may include a groove formed on an outer circumferential surface so that the motion wire is wound therearound, the groove may have a length in a direction in which the motion wire is wound, and the length of the groove may be equal to or greater than a maximum forward motion distance over which the operation member is movable forward.

In the present embodiment, the jaw may include a first jaw and a second jaw facing each other and configured to perform an open motion and a close motion, the first jaw may include an accommodation part configured to accommodate a plurality of staples, and the motion belt may be disposed in a region of the first jaw opposite to a surface on which the accommodation part is formed.

According to an embodiment, a surgical instrument includes the end tool described above, a manipulation part configured to control an operation of the end tool, and a connection part configured to connect the manipulation part to the end tool.

In the present embodiment, the end tool may be configured to perform a yaw motion around one shaft and perform a pitch motion around another shaft different from the one shaft.

Other aspects, features, and advantages of the disclosure will become better understood through the accompanying drawings, the appended claims, and the detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic perspective view of a surgical instrument according to an embodiment;

FIG. 2 is a schematic perspective view for describing an end tool of FIG. 1;

FIG. 3 is a perspective view for describing a first jaw of the end tool of FIG. 2;

FIG. 4 is a rear view of the first jaw of the end tool of FIG. 2, when viewed from below;

FIG. 5 is a perspective view illustrating an operation member of the end tool of FIG. 2;

FIG. 6 is a rear view of the operation member of FIG. 5, when viewed from below;

FIG. 7 is a side view for describing the operation member of FIG. 5;

FIG. 8 is a schematic perspective view for describing the operation member and a reciprocating moving part of the end tool of FIG. 2;

FIG. 9 is a perspective view illustrating a transmission gear of FIG. 8;

FIG. 10 is a plan view illustrating the reciprocating moving part;

FIG. 11 is a perspective view illustrating the reciprocating moving part;

FIG. 12 is a diagram for describing the operation of the operation member of the end tool of FIG. 2;

FIG. 13 is a diagram for describing the reciprocating moving part, a driving gear, and the transmission gear, which are related to the operation of the operation member of FIG. 12;

FIG. 14 is a diagram for describing another operation of the operation member of the end tool of FIG. 2;

FIG. 15 is a diagram for describing the reciprocating moving part, the driving gear, and the transmission gear, which are related to the operation of the operation member of FIG. 14;

FIG. 16 is a perspective view illustrating a first jaw and a cartridge of the surgical instrument of FIG. 1;

FIGS. 17 and 18 are cross-sectional views illustrating the overall stapling motion of the end tool of the surgical instrument of FIG. 1;

FIG. 19 is a diagram illustrating a modification of the end tool of FIG. 2;

FIG. 20 is a schematic perspective view for describing an end tool of a surgical instrument according to another embodiment;

FIGS. 21 and 22 are schematic perspective views for describing an operation member and a reciprocating moving part of the end tool of FIG. 20;

FIG. 23 is a plan view illustrating a driving gear of the end tool of FIG. 20;

FIG. 24 is a perspective view illustrating the reciprocating moving part of the end tool of FIG. 20;

FIGS. 25 and 26 are diagrams for describing the operation of the operation member of the end tool of FIG. 20;

FIGS. 27 and 28 are diagrams illustrating a modification of the end tool of FIG. 20;

FIG. 29 is a schematic perspective view for describing an end tool of a surgical instrument according to another embodiment;

FIG. 30 is a rear view of the end tool of FIG. 29, when viewed from below;

FIG. 31 is a perspective view for describing a belt part and a driving module of the end tool of FIG. 29;

FIG. 32 is a perspective view schematically illustrating the belt part and the driving module of FIG. 31;

FIG. 33 is a diagram illustrating a modification of the belt part of FIG. 32;

FIG. 34 is a schematic perspective view for describing an end tool of a surgical instrument according to another embodiment;

FIG. 35 is a perspective view of the end tool of FIG. 34, when viewed from another direction;

FIG. 36 is a plan view for describing a motion wire and a driving module of the end tool of FIG. 34;

FIG. 37 is a perspective view for describing the motion wire and the driving module of the end tool of FIG. 34;

FIG. 38 is a schematic perspective view of a surgical instrument according to another embodiment;

FIG. 39 is a side view of the surgical instrument of FIG. 38; and

FIG. 40 is a diagram for schematically describing the operation concept of the surgical instrument of FIG. 38.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

As the present description allows for various changes and numerous embodiments, certain embodiments will be illustrated in the drawings and described in detail in the written description. Effects and features of the disclosure, and methods of achieving them will be clarified with reference to embodiments described below in detail with reference to the drawings. However, the disclosure is not limited to the following embodiments and may be embodied in various forms.

Hereinafter, embodiments will be described in detail with reference to the accompanying drawings. When describing embodiments with reference to the accompanying drawings, the same or corresponding elements are denoted by the same reference numerals, and redundant descriptions thereof are omitted.

It will be understood that although the terms “first,” “second,” etc. may be used herein to describe various elements, these elements should not be limited by these terms. These terms are only used to distinguish one element from another.

The singular forms as used herein are intended to include the plural forms as well unless the context clearly indicates otherwise.

It will be further understood that the terms “include” and/or “comprise” used herein specify the presence of stated features or elements, but do not preclude the presence or addition of one or more other features or elements.

Also, sizes of elements in the drawings may be exaggerated or reduced for convenience of explanation. For example, because size and thickness of elements in the drawings are arbitrarily illustrated for convenience of explanation, the disclosure is not limited thereto.

The x-axis, the y-axis, and the z-axis are not limited to three axes of the rectangular coordinate system and may be interpreted in a broader sense. For example, the x-axis, the y-axis, and the z-axis may be perpendicular to one another or may represent different directions that are not perpendicular to one another.

When a certain embodiment is implemented differently, a specific process sequence may be performed differently from a sequence described herein. For example, two consecutively described processes may be performed substantially at the same time or performed in an order opposite to the stated order.

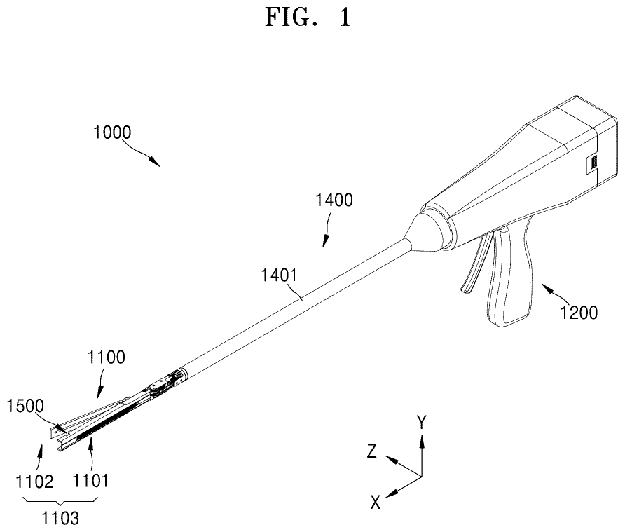

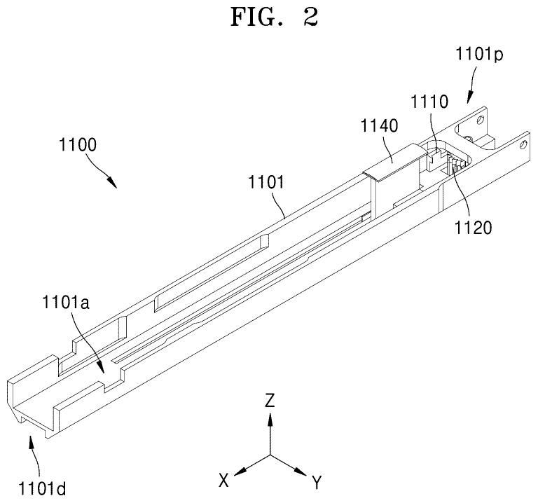

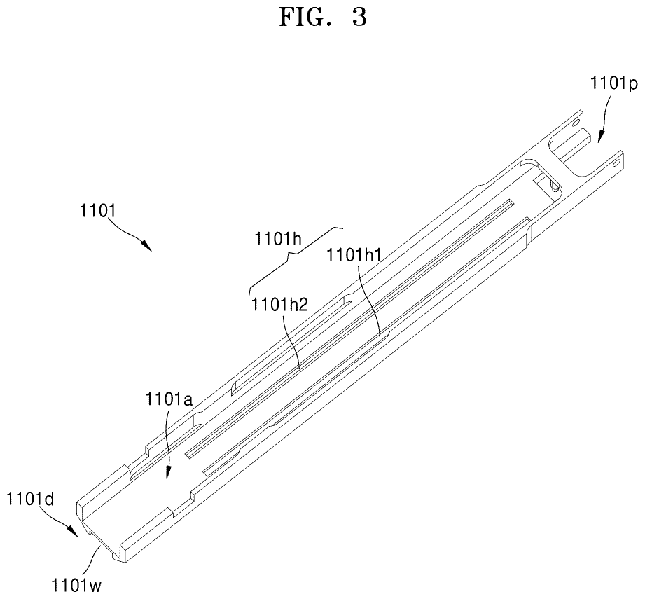

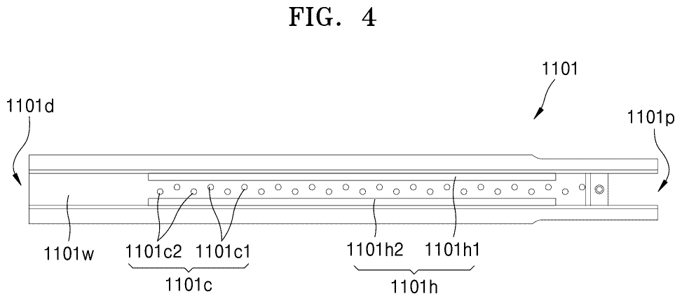

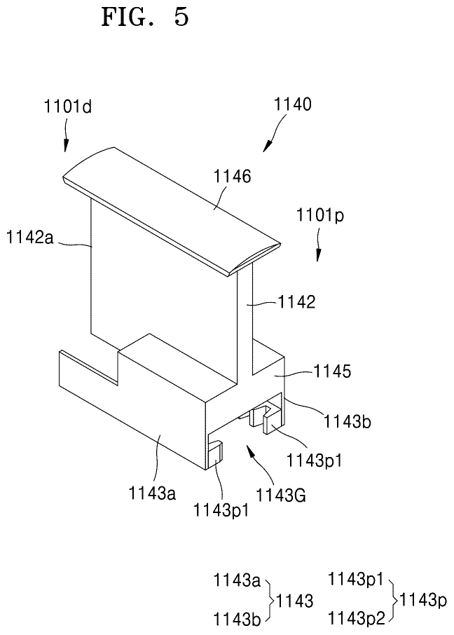

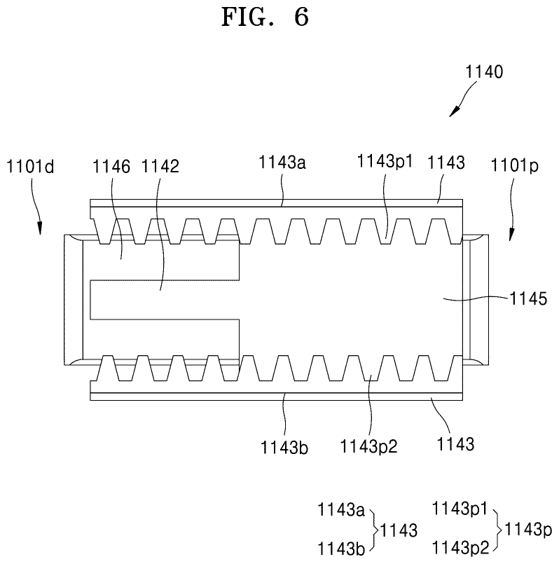

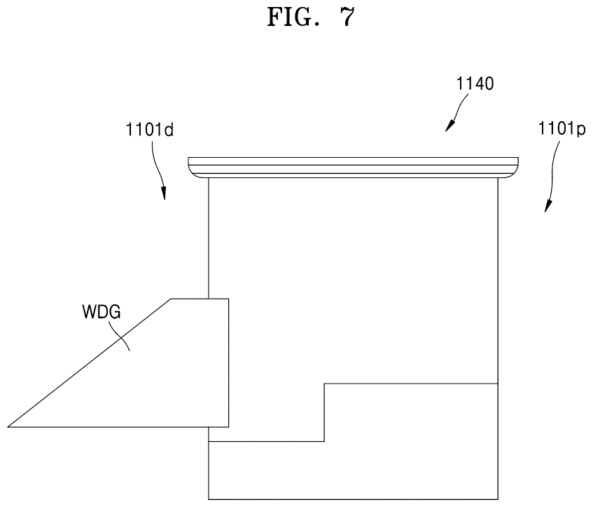

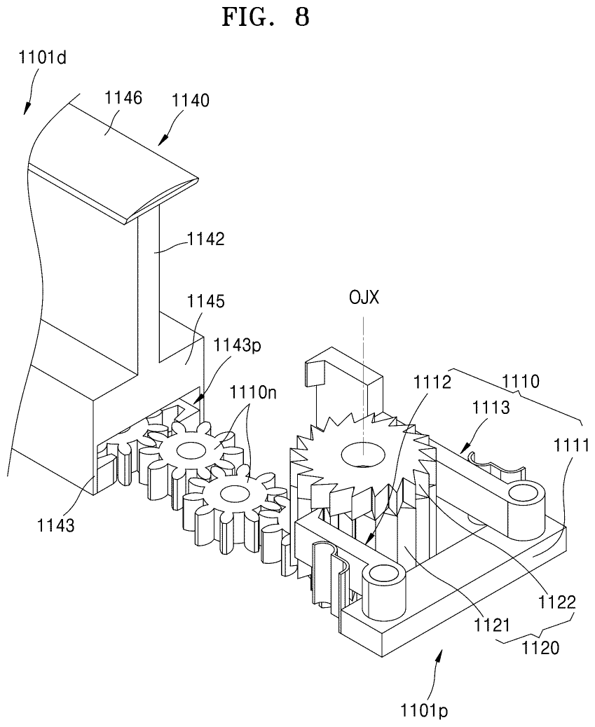

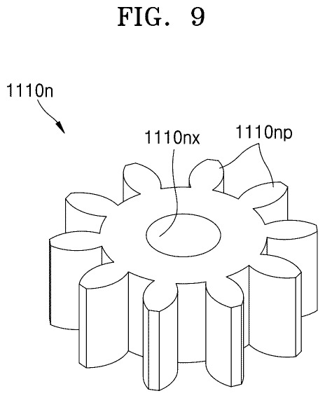

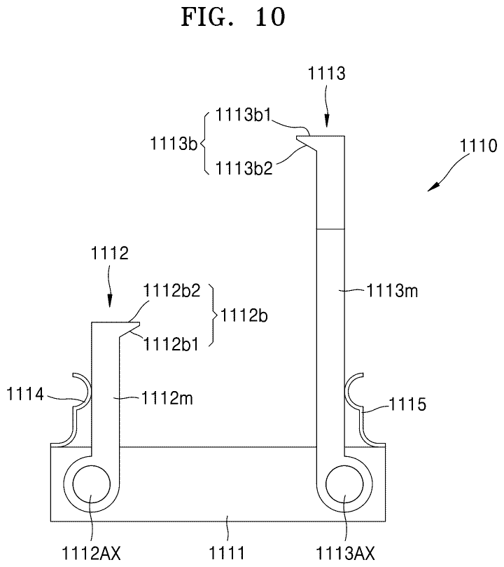

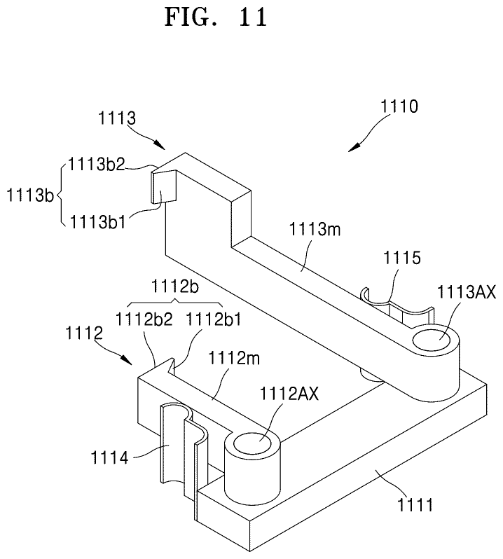

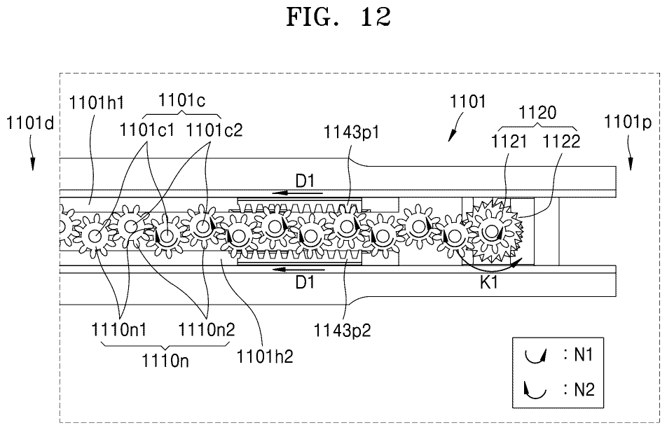

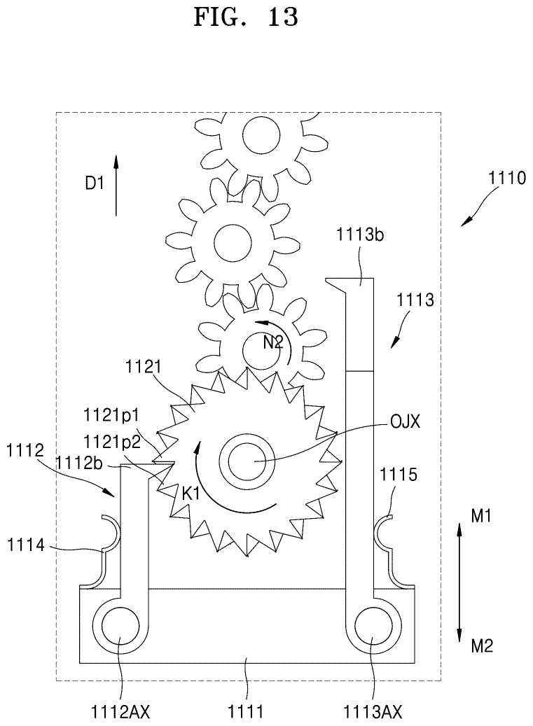

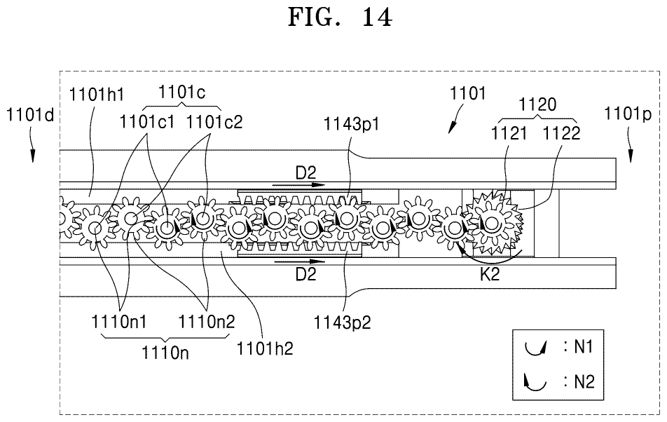

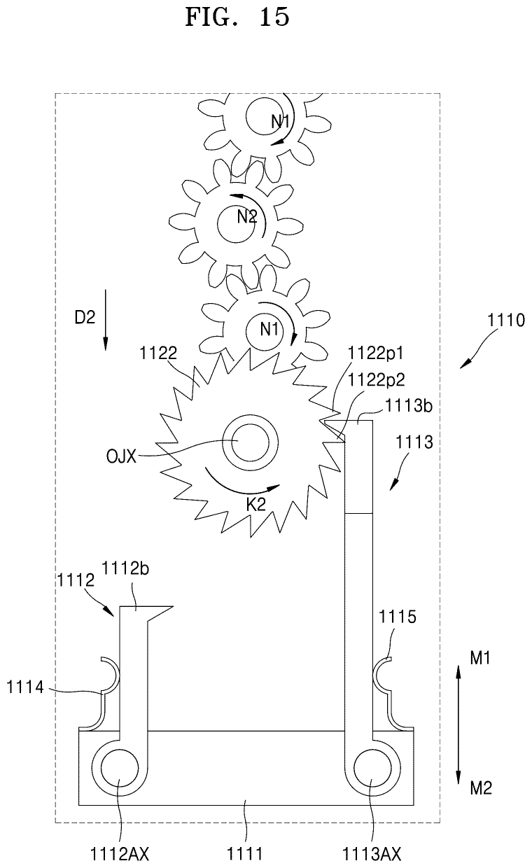

FIG. 1 is a schematic perspective view of a surgical instrument according to an embodiment. FIG. 2 is a schematic perspective view for describing an end tool of FIG. 1. FIG. 3 is a perspective view for describing a first jaw of the end tool of FIG. 2. FIG. 4 is a rear view of the first jaw of the end tool of FIG. 2, when viewed from below. FIG. 5 is a perspective view illustrating an operation member of the end tool of FIG. 2. FIG. 6 is a rear view of the operation member of FIG. 5, when viewed from below. FIG. 7 is a side view for describing the operation member of FIG. 5. FIG. 8 is a schematic perspective view for describing the operation member and a reciprocating moving part of the end tool of FIG. 2. FIG. 9 is a perspective view illustrating a transmission gear of FIG. 8. FIG. 10 is a plan view illustrating the reciprocating moving part. FIG. 11 is a perspective view illustrating the reciprocating moving part. FIG. 12 is a diagram for describing the operation of the operation member of the end tool of FIG. 2. FIG. 13 is a diagram for describing the reciprocating moving part, a driving gear, and the transmission gear, which are related to the operation of the operation member of FIG. 12. FIG. 14 is a diagram for describing another operation of the operation member of the end tool of FIG. 2. FIG. 15 is a diagram for describing the reciprocating moving part, the driving gear, and the transmission gear, which are related to the operation of the operation member of FIG. 14.

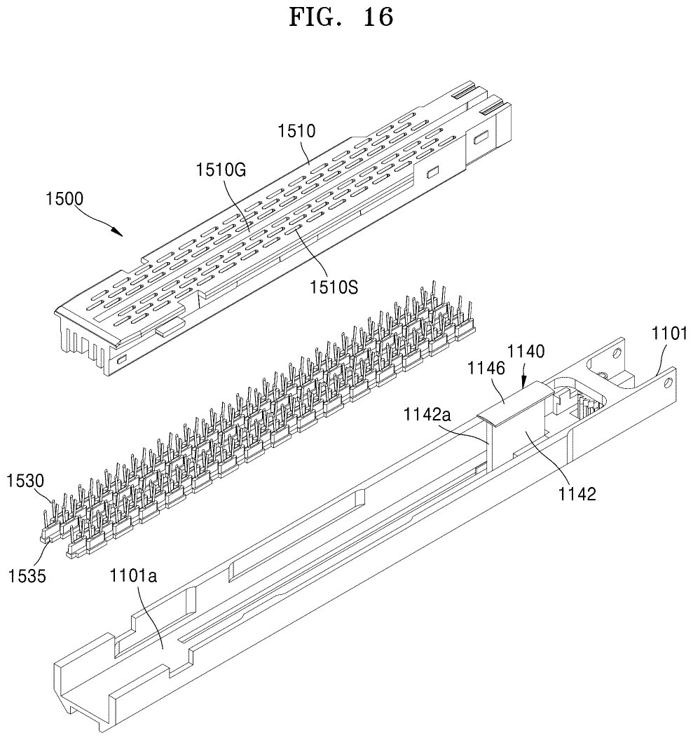

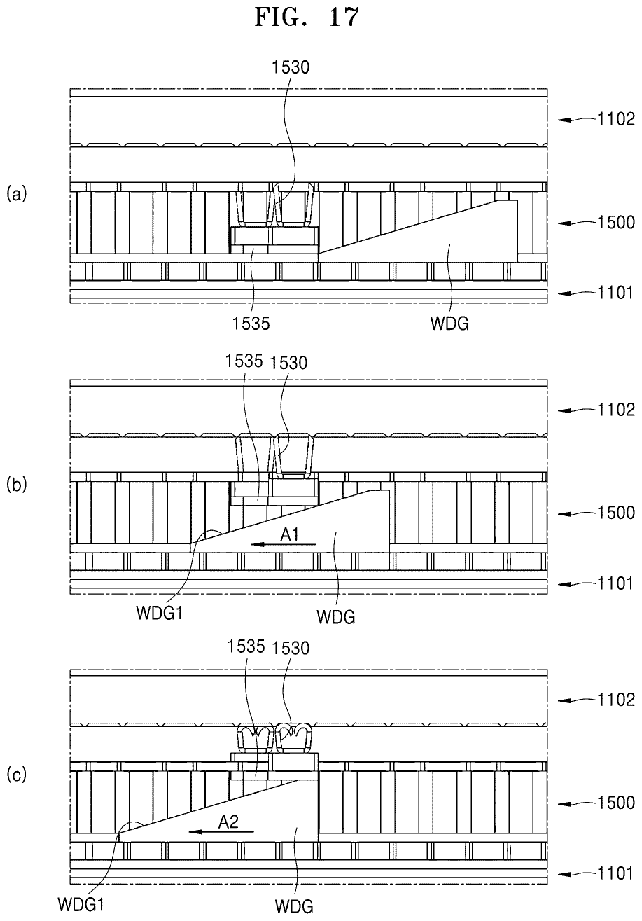

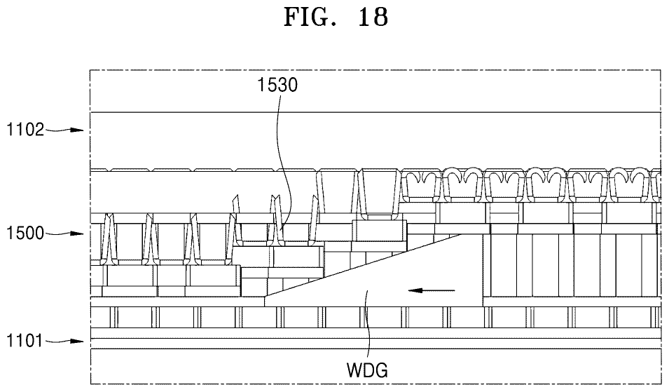

FIG. 16 is a perspective view illustrating a first jaw and a cartridge of the surgical instrument of FIG. 1. FIGS. 17 and 18 are cross-sectional views illustrating the overall stapling motion of the end tool of the surgical instrument of FIG. 1.

A surgical instrument 1000 according to the present embodiment may include an end tool 1100, a manipulation part 1200, and a connection part 1400.

The connection part 1400 may be formed in the shape of a hollow shaft, and one or more wires and electric wires may be accommodated therein. The manipulation part 1200 may be coupled to one end portion of the connection part 1400, and the end tool 1100 may be coupled to the other end portion of the connection part 1400, so that the connection part 1400 may serve to connect the manipulation part 1200 to the end tool 1100.

The manipulation part 1200 is formed at one end portion of the connection part 1400 and provided as an interface to be directly controlled by a medical doctor, for example, a tongs shape, a stick shape, a lever shape, or the like, and when the medical doctor controls the manipulation part 1200, an end tool (not shown), which is connected to the interface and inserted into the body of a surgical patient, performs a certain motion, thereby performing surgery. Although FIG. 1 illustrates that the manipulation part 1200 is formed in the shape of a handle that allows a finger to be in close contact therewith to perform one or more operations, for example, pulling or pushing, but the concept of the disclosure is not limited thereto, and various types of manipulation parts that are connectable to the end tool 1100 and manipulate the end tool 1100 may be possible.

The end tool 1100 is formed at the other end portion of the connection part 1400 and is inserted into a surgical site to perform motions necessary for surgery. As an example of the end tool 1100 described above, a pair of jaws 1103 for performing a gripping motion may be used. However, the concept of the disclosure is not limited thereto, and various devices for performing surgery may be used as the end tool 1100. For example, a configuration of a cantilever cautery may also be used as the end tool 1100. The end tool 1100 is connected to the manipulation part 1200 by a power transmission part (not shown) (e.g., a wire, etc.) and receives a driving force of the manipulation part 1200 through the power transmission part to perform a motion necessary for surgery, such as gripping, cutting, suturing, or the like.

Hereinafter, the end tool 1100 of the surgical instrument 1000 of FIG. 2 is described in more detail.

FIG. 2 is a schematic perspective view for describing the end tool of FIG. 1.

FIG. 3 is a perspective view for describing a first jaw of the end tool of FIG. 2.

FIG. 4 is a rear view of the first jaw of the end tool of FIG. 2, when viewed from below.

The end tool 1100 may include a jaw 1103, a reciprocating moving part 1110, and a plurality of transmission gears 1110n.

Referring to FIG. 2, the jaw 1103 of the end tool 1100 is illustrated. For convenience of explanation, a first jaw 1101 is mainly illustrated, and a second jaw 1102, a cartridge 1500, and the like are omitted.

The end tool 1100 of the present embodiment may include the jaw 1103. The jaw 1103 may perform various functions, for example, a gripping motion. As a specific example, the jaw 1103 may include a pair of jaws, that is, the first jaw 1101 and the second jaw 1102. Each of the first jaw 1101 and the second jaw 1102 or an element encompassing the first jaw 1101 and the second jaw 1102 may be referred to as the jaw 1103.

The first jaw 1101 may be formed in the shape of an elongated bar as a whole, a cartridge (1500 of FIG. 1) may be accommodated in the first jaw 1101 at a distal end 1101d side, and a rotation shaft may be disposed at a proximal end 1101p so that the first jaw 1101 is rotatable around the rotation shaft.

The first jaw 1101 may be formed entirely in the form of a hollow box, of which one surface (upper surface) is removed, so that a cartridge accommodation part 1101a capable of accommodating the cartridge 1500 may be formed inside the first jaw 1101. That is, the first jaw 1101 may be formed in an approximately “U” shape in cross section.

One or more first guide grooves 1101h may be formed on the bottom surface of the first jaw 1101, that is, on the bottom surface facing the upper open region in which one surface is removed. Specifically, the first guide groove 1101h may be configured to guide a linear motion of an operation member 1140, which will be described later.

The first guide groove 1101h may have a plurality of grooves. For example, the first guide groove 1101h may include a first side groove 1101h1 and a second side groove 1101h2 that may be apart from each other. The first side groove 1101h1 and the second side groove 1101h2 may correspond to a side portion 1143 of the operation member 1140. Details thereof are described later. The first side groove 1101h1 and the second side groove 1101h2 may have a shape in which one region is removed and have a length that extends in the longitudinal direction of the first jaw 1101.

Referring to FIGS. 3 and 4, the first jaw 1101 may include an extension groove 1101w in a region facing the back surface thereof. The extension groove 1101w may be formed on the back surface of the first jaw 1101, that is, on the surface opposite to the surface on which the cartridge accommodation part 1101a accommodating the cartridge is formed.

The extension groove 1101w may be formed to overlap the movement path of the operation member 1140 and may have a certain length along the longitudinal direction of the first jaw 1101. For example, the extension groove 1101w may be formed to have the same length as the length of the first jaw 1101.

The extension groove 1101w may be formed to overlap the first side groove 1101h1 and the second side groove 1101h2 of the first guide groove 1101h. Accordingly, when the operation member 1140 is moved in one direction, at least one region of the operation member 1140, for example, one region of the side portion 1143 of the operation member 1140 may be moved through the first side groove 1101h1 and the second side groove 1101h2 while corresponding to the extension groove 1101w.

The extension groove 1101w may be formed to have a width greater than a width of each of the transmission gears 1110n so that at least the plurality of transmission gears 1110n may be disposed therein. Accordingly, the plurality of transmission gears 1110n may perform a rotational motion while corresponding to the extension groove 1101w.

To facilitate the rotational motion, a plurality of rotation shaft 1101c may be formed to protrude on one surface of the extension groove 1101w. The transmission gears 1110n may respectively correspond to the rotation shafts 1101c. For example, the plurality of transmission gears 1110n may be respectively fitted into the protruding shapes of the rotation shafts 1101c and perform a rotational motion.

The rotation shafts 1101c may be disposed along the longitudinal direction of the extension groove 1101w. For example, the rotation shafts 1101c may correspond to the first side groove 1101h1 and the second side groove 1101h2 of the first guide groove 1101h. As a specific example, the rotation shafts 1101c may be formed between the first side groove 1101h1 and the second side groove 1101h2.

In addition, as a specific example, the rotation shaft 1101c may include a first shaft 1101c1 and a second shaft 1101c2. The first shaft 1101c1 and the second shaft 1101c2 may be formed at misaligned positions with respect to the longitudinal direction of the extension groove 1101w. For example, the first shaft 1101c1 may be close to one side surface with respect to the width direction of the extension groove 1101w, and the second shaft 1101c2 may be close to the other side surface with respect to the width direction of the extension groove 1101w. In addition, it may also be said that the first shaft 1101c1 may be disposed close to the first side groove 1101h1 of the first guide groove 1101h, and the second shaft 1101c2 may be disposed close to the second side groove 1101h2 of the first guide groove 1101h.

In addition, as an alternative embodiment, the first shaft 1101c1 and the second shaft 1101c2 may each be provided in plurality. For example, the first shaft 1101c1 and the second shaft 1101c2 may be alternated multiple times.

Through such an arrangement, the transmission gears 1110n may be disposed to be misaligned with each other. For example, the first transmission gear 1110n1 and the second transmission gear 1110n2 may be alternately disposed to be misaligned with each other (see FIG. 12).

The operation member 1140 is described in detail.

FIG. 5 is a perspective view illustrating the operation member of the end tool of FIG. 2. FIG. 6 is a rear view of the operation member of FIG. 5, when viewed from below. FIG. 7 is a side view for describing the operation member of FIG. 5.

The operation member 1140 may include a main body 1145, a blade 1142, and a side portion 1143. Meanwhile, the operation member 1140 may be used together with a wedge WDG (see FIGS. 7, 17, and 18). For example, the wedge WDG (see FIGS. 7, 17, and 18) may be prepared separately from the operation member 1140 and then disposed adjacent to the operation member 1140 in the first jaw 1101. In addition, as another example, the operation member 1140 and the wedge may be integrally formed with each other. In the present specification, for convenience of explanation, the description and illustration are made on the assumption that the operation member 1140 and the wedge are separately prepared.

The wedge may be disposed on at least one side of the main body 1545 and may be formed to have a certain inclined surface. That is, the wedge may be formed to be inclined to a certain degree in the extension direction of the end tool 1100. In other words, the height of the proximal end 1101p of the first jaw 1001 may be formed to be higher than the height of the distal end 1101d.

The wedge may be formed to be in sequential contact with a withdrawal member (1535 of FIG. 16) or a plurality of staples (1530 of FIG. 16) and may sequentially push and raise the staples 1530. As illustrated in FIGS. 17 and 18, which will be described later, while the operation member 1140 is moved toward the distal end 1101d, the operation member 1140 may sequentially push and raise the staples 1530 and withdraw the staples 1530 to the outside of the cartridge 1500.

The blade 1142 may have a shape that protrudes from the main body 1145, and an edge portion 1142a that is sharp and cuts tissue is formed in one region of the blade 1142. As at least a portion of the edge portion 1142a is withdrawn to the outside of the first jaw 1101 and the cartridge 1500, tissue disposed between the first jaw 1101 and the second jaw 1102 may be cut. At least one region of the blade 1142 and the edge portion 1142a may always be withdrawn to the outside of the first jaw 1101. In addition, as another example, at least one region of the blade 1142 and the edge portion 1142a may be accommodated in the first jaw 1101 or the cartridge 1500 and may be then withdrawn to the outside of the first jaw 1101 only when the operation member 1140 is moved along the longitudinal direction.

As an alternative embodiment, a clamp 1146 may be further formed in one region of the blade 1142, and the width of the clamp 1146 may be formed to be at least greater than the width of the blade 1142. The clamp 1146 may be fitted into a groove formed along the longitudinal direction of the second jaw 1102, and the clamp 1146 may be moved while passing through the groove and being disposed on or in contact with the upper surface of the second jaw 1102. Accordingly, when the operation member 1140 is moved, the clamp 1146 may apply a force in a direction to bring the second jaw 1102 closer to the first jaw 1101.

As a result, a motion in which the second jaw 1102 is brought closer to the first jaw 1101 through the clamp 1146 when the operation member 1140 is moved in a direction from the proximal end 1101p to the distal end 1101d of the first jaw 1101, that is, a closing motion of the jaw 1103, may be naturally implemented.

The side portion 1143 may be formed to have a height in a region of the main body 1145 in a direction opposite to the protruding direction of the blade 1142. The side portion 1143 may have a protruding shape facing the bottom surface of the first jaw 1101. For example, the side portion 1143 may include a first side portion 1143a and a first side portion 1143a respectively protruding from both sides of the main body 1145 in the width direction.

The first side portion 1143a and the second side portion 1143b may be spaced apart from each other, and a separation space 1143G may be formed between the first side portion 1143a and the second side portion 1143b at a location including a region that overlaps the center of the main body 1145 and the blade 1142.

The first side portion 1143a and the second side portion 1143b may be disposed in parallel to face each other. For example, the first side portion 1143a and the second side portion 1143b may be respectively formed to correspond to the first side groove 1101h1 and the second side groove 1101h2 of the first guide groove 1101h of the first jaw 1101. The operation member 1140 may perform a forward motion while the first side portion 1143a and the second side portion 1143b are respectively fitted into the first side groove 1101h1 and the second side groove 1101h2. To this end, the width of each of the first side groove 1101h1 and the second side groove 1101h2 may be formed to be greater than the width of each of the first side portion 1143a and the second side portion 1143b.

A rack member 1143p having a plurality of concavo-convex portions may be formed on the side portion 1143 and may be formed on the first side portion 1143a and the second side portion 1143b.

For example, the rack member 1143p may have a plurality of members. As a specific example, the rack member 1143p may include a first rack member 1143p1 and a second rack member 1143p2.

The first rack member 1143p1 and the second rack member 1143p2 may be disposed to be spaced apart from each other and face each other, and the concavo-convex portion of the first rack member 1143p1 and the concavo-convex portion of the second rack member 1143p2 may be disposed to face each other.

For example, the first rack member 1143p1 and the second rack member 1143p2 may be respectively disposed to correspond to the first side portion 1143a and the second side portion 1143b in the separation space 1143G of the operation member 1140.

As a specific example, the first rack member 1143p1 of the rack member 1143p may be disposed to correspond to the inner surface of the first side portion 1143a, and the second rack member 1143p2 may be disposed to correspond to the inner surface of the second side portion 1143b.

For example, when the operation member 1140 is disposed in the first jaw 1101, the first side portion 1143a and the second side portion 1143b of the side portion 1143 may pass through the first side groove 1101h1 and the second side groove 1101h2 of the first guide groove 1101h of the first jaw 1101 and extend to the extension groove 1101w of the first jaw 1101. At this time, the first rack member 1143p1 and the second rack member 1143p2 of the rack member 1143p may be respectively disposed to correspond to the inner surface of the first side portion 1143a and the inner surface of the second side portion 1143b extending to the extension groove 1101w.

At this time, the rack member 1143p may be formed to be movable integrally with at least the operation member 1140. For example, the first rack member 1143p1 and the second rack member 1143p2 may be respectively formed to be coupled to the inner surface of the first side portion 1143a and the inner surface of the second side portion 1143b of the operation member 1140. As another example, the first rack member 1143p1 may be integrally formed with the first side portion 1143a, and the second rack member 1143p2 may be integrally formed with the second side portion 1143b.

Through such a configuration, when a force is applied to the rack member 1143p, for example, when a force in one direction is applied along the longitudinal direction of the first jaw 1101, the rack member 1143p may perform a motion and the operation member 140 may perform a motion together with the rack member 1143p.

Specifically, as the transmission gear 1110n performs a rotational motion, teeth of the transmission gear 1110n are coupled to the concavo-convex portion of the rack member 1143p to transmit a force. The force causes the rack member 1143p and the operation member 1140 to move. Details thereof are described later.

The reciprocating moving part 1110, the driving gear 1120, and the like are described in more detail.

FIG. 8 is a schematic perspective view for describing the operation member and a reciprocating moving part of the end tool of FIG. 2; FIG. 9 is a perspective view illustrating the transmission gear of FIG. 8. FIG. 10 is a plan view illustrating the reciprocating moving part. FIG. 11 is a perspective view illustrating the reciprocating moving part.

Referring to FIG. 8, the reciprocating moving part 1110 may be configured to perform a reciprocating linear motion in both directions toward the proximal end 1101p and the distal end 1101d of the first jaw 1101. During the linear motion, the driving gear 1120 connected to the reciprocating moving part 1110 may perform a motion and the transmission gears 1110n may perform a rotational motion according to the motion of the driving gear 1120. Due to the rotational motion, the rack member 1143p may be moved and the operation member 1140 may be moved together with the rack member 1143p. The driving gear 1120 may be disposed to be rotatable around a rotation shaft OJX. A first driving gear 1121 and a second driving gear 1122, which will be described later, may be disposed to be rotatable around the same rotation shaft OJX.

Referring to FIG. 9, the transmission gear 1110n has a circular shape and may have a groove 1101nx corresponding to the rotation shaft (1101c of FIG. 4) at the center thereof, and a plurality of teeth 1101np may be formed on the outer circumferential surface thereof.

Referring to FIGS. 8 to 11, the reciprocating moving part 1110 may include a main body 1111, a first contact part 1112, and a second contact part 1113.

The main body 1111 may have an elongated bar shape and may form the base of the reciprocating moving part 1110 in various shapes, such as a straight line or a curve. The width and thickness of the main body 1111 may be appropriately determined to withstand a load when transmitting a force in contact with the driving gear 1120 through the motions of the first contact part 1112 and the second contact part 1113.

The first contact part 1112 and the second contact part 1113 may be configured to be connected to the main body 1111 while being spaced apart from each other. The first contact part 1112 and the second contact part 1113 may each be formed to have a length from the main body 1111 along the longitudinal direction of the first jaw 1101 and may extend in a direction toward the operation member 1140.

The first contact part 1112 and the second contact part 1113 may be formed to have different lengths. For example, the length of the first contact part 1112 may be less than the length of the second contact part 1113.

The first contact part 1112 may include an extension part 1112m connected to the main body 1111 and extending from the main body 1111 in the longitudinal direction of the first contact part 1112, and a protrusion part 1112b formed at one end of the extension part 1112m. The protrusion part 1112b may have a shape that protrudes toward the inside of the first contact part 1112, that is, the second contact part 1113. The protrusion part 1112b may be formed to be in contact with the first driving gear 1121 of the driving gear 1120. The protrusion part 1112b may include a first surface 1112b1 and a second surface 1112b2. The first surface 1112b1 of the protrusion part 1112b may have an inclined surface. For example, the first surface 1112b1 of the protrusion part 1112b may be inclined with respect to the extension part 1112m. The second surface 1112b2 of the protrusion part 1112b may be a vertical or substantially vertical surface. The first surface 1112b1 of the protrusion part 1112b may be disposed behind the second surface 1112b2 of the protrusion part 1112b. For example, the first surface 1112b1 of the protrusion part 1112b may be disposed closer to the main body 1111 than the second surface 1112b2 of the protrusion part 1112b.

Accordingly, when the first contact part 1112 comes into contact with the first driving gear 1121 of the driving gear 1120, the first contact part 1112 may be coupled to the ratchet of the first driving gear 1121 and cause the first driving gear 1121 to move in one direction. Details thereof are described later.

The second contact part 1113 may include an extension part 1113m connected to the main body 1111 and extending from the main body 1111 in the longitudinal direction of the second contact part 1113, and a protrusion part 1113b formed at one end of the extension part 1113m. The protrusion part 1113b may have a shape that protrudes toward the inside of the second contact part 1113, that is, the first contact part 1112. The protrusion part 1113b may be formed to be in contact with the second driving gear 1122 of the driving gear 1120. The protrusion part 1113b may include a first surface 1113b1 and a second surface 1113b2. The first surface 1113b1 of the protrusion part 1113b may have an inclined surface. For example, the first surface 1113b1 of the protrusion part 1113b may be inclined with respect to the extension part 1113m. The second surface 1113b2 of the protrusion part 1113b may be a vertical or substantially vertical surface. The second surface 1113b2 of the protrusion part 1113b may be disposed behind the first surface 1113b1 of the protrusion part 1113b. For example, the first surface 1113b1 of the protrusion part 1113b may be disposed closer to the main body 1111 than the second surface 1113b2 of the protrusion part 1113b.

Accordingly, when the second contact part 1113 comes into contact with the second driving gear 1122 of the driving gear 1120, the second contact part 1113 may be coupled to the ratchet of the second driving gear 1122 and cause the second driving gear 1122 to move in one direction. Details thereof are described later.

The protrusion part 1113b of the second contact part 1113 may be located higher than the protrusion part 1112b of the first contact part 1112 with respect to the main body 1111. Accordingly, when the first driving gear 1121 and the second driving gear 1122 of the driving gear 1120 are sequentially stacked from below, the protrusion part 1112b of the first contact part 1112 may be only in contact with the first driving gear 1121 without being in contact with the second driving gear 1122 (see FIG. 13). In addition, the protrusion part 1113b of the second contact part 1113 may be only in contact with the second driving gear 1122 without being in contact with the first driving gear 1121n (see FIG. 15), so that the driving of the driving gear 1120 may be precisely controlled.

In the present embodiment, a case where the first contact part 1112 has the protrusion part 1112b and the second contact part 1113 has the protrusion part 1113b has been described. However, as an alternative embodiment, the first contact part 1112 or the second contact part 1113 may have a plurality of protrusion parts.

As an alternative embodiment, the first contact part 1112 and the second contact part 1113 may respectively have a first rotation shaft 1112AX and a second rotation shaft 1113AX so as to perform a rotational motion while being connected to the main body 1111.

In addition, a first elastic member 1114 and a second elastic member 1115 respectively capable of providing an elastic force to the first contact part 1112 and the second contact part 1113 may be disposed on the main body 1111.

The first elastic member 1114 may provide an elastic force to apply a force in an opposite direction when the first contact part 1112 performs a rotational motion outward through the first rotation shaft 1112AX (a motion in a direction away from the second contact part). Accordingly, when the first contact part 1112 comes into contact with the first driving gear 1121 of the driving gear 1120 and applies a force thereto, the force may be effectively sequentially applied to the ratchets 1121p of the first driving gear 1121.

The second elastic member 1115 may provide an elastic force to apply a force in an opposite direction when the second contact part 1113 performs a rotational motion outward through the second rotation shaft 1113AX (a motion in a direction away from the first contact part). Accordingly, when the second contact part 1113 comes into contact with the second driving gear 1122 of the driving gear 1120 and applies a force thereto, the force may be effectively sequentially applied to the ratchets 1122p of the second driving gear 1122.

The reciprocating moving part 1110 may be configured to receive a driving force from a driving part and perform a linear motion, for example, a forward motion M1 and a backward motion M2 in FIG. 13. As a specific example, rotation of a motor part in one direction may be converted into a bidirectional linear motion through connection of various members, such as pulleys and links, and the bidirectional linear motion may be then transmitted to the reciprocating moving part 1110. In addition, the linear motion of the driving part may be transmitted to the reciprocating moving part 1110 in various other methods. For example, the linear motion of the driving part may be directly transmitted to the reciprocating moving part 1110.

For example, the reciprocating moving part 1110 may perform the forward motion and the backward motion at one or more points. As a specific example, the reciprocating moving part 1110 may perform the forward motion and the backward motion at two points. In this case, the operation member 1140 may easily perform the forward motion and the backward motion through the reciprocating moving part 1110. A first position among the two points is a position of a point where the first contact part 1112 of the reciprocating moving part 1110 comes into contact with the first driving gear 1121 of the driving gear 1120 (see FIG. 13), and a second position that is the other point is a position of a point where the second contact part 1113 of the reciprocating moving part 1110 comes into contact with the second driving gear 1122 of the driving gear 1120 (see FIG. 15). That is, the second position (see FIG. 15) of the reciprocating moving part 1110 may be located behind the first position of the reciprocating moving part 1110 (see FIG. 13). In addition, it may also be said that the first position (see FIG. 13) and the second position (see FIG. 15) of the reciprocating moving part 1110 may be respectively positions defined with distances in order with respect to a direction from the distal end 1101d to the proximal end 1101p of the first jaw 110. It may also be said that the first position (see FIG. 13) of the reciprocating moving part 1110 may be a position disposed closer to the operation member 1140 than the second position (see FIG. 15) of the reciprocating moving part 1110. The position of the reciprocating moving part 1110 may be changed in various methods. For example, the position of the reciprocating moving part 1110 may be determined by transmitting a force through one or more driving wires and pulling or pushing the reciprocating moving part 1110.

In addition, as an alternative embodiment, the reciprocating moving part 1110 may perform the forward motion and the backward motion only at one point (e.g., the first position (see FIG. 13)). In this case, the operation member 1140 may perform only the forward motion through the reciprocating moving part 1110.

As a specific example, among the forward motion of the operation member 1140 through the first contact part 1112 of the reciprocating moving part 1110 (see FIGS. 12 and 13) and the backward motion of the operation member 1140 through the second contact part 1113 (FIGS. 14 and 15), which will be described later, only the forward motion of the operation member 1140 through the first contact part 1112 (see FIGS. 12 and 13) may be performed, and in this case, the second contact part 1113 may be omitted.

The motion of the operation member 1140 is described.

FIG. 12 is a diagram for describing the operation of the operation member of the end tool of FIG. 2. FIG. 13 is a diagram for describing the reciprocating moving part, the driving gear, and the transmission gear, which are related to the operation of the operation member of FIG. 12. FIG. 14 is a diagram for describing another operation of the operation member of the end tool of FIG. 2. FIG. 15 is a diagram for describing the reciprocating moving part, the driving gear, and the transmission gear, which are related to the operation of the operation member of FIG. 14.

The motion of the operation member 1140 is described with reference to FIGS. 8, 12, and 13.

Referring to FIG. 13, the reciprocating moving part 1110 may perform the forward motion M1 and the backward motion M2 as a whole. At this time, the reciprocating moving part 1110 is in the first position, that is, the position where the first contact part 1112 is in contact with the first driving gear 1121 of the driving gear 1120.

In this case, the first driving gear 1121 may be rotated in one direction K1, for example, a clockwise direction, according to the forward motion M1 of the first contact part 1112. At this time, the second contact part 1113 may be spaced apart from the first driving gear 1121 and the second driving gear 1122.

Specifically, the first driving gear 1121 may have the form of a ratchet. For example, the first driving gear 1121 may include a plurality of teeth 1121p1 and 1121p2. As a specific example, the first driving gear 1121 may include the first teeth 1121p1 each having an inclined surface and a vertical or substantially vertical surface.

The vertical or substantially vertical second surface 1112b2 of the protrusion part 1112b of the first contact part 1112 may meet and engage with the vertical or substantially vertical surface of the first tooth 1121p1 of the first driving gear 1121, and the force of the forward motion M1 through the contact part 1112 may be transmitted so that the first driving gear 1121 is rotated by the first tooth 1121p1 of the first driving gear 1121.

During the backward motion M2 of the first contact part 1112, the first surface 1112b1, which is the inclined surface of the protrusion part 1112b of the first contact part 1112, may correspond to the inclined surface of the second tooth 1121p2 of the first driving gear 1121, the protrusion part 1112b may slide over the inclined surface of the second tooth 1121p2, and the first driving gear 1121 may maintain a stationary state.

At this time, the first contact part 1112 may be rotated while being pushed outward and may come into close contact with the first driving gear 1121 again through the first elastic member 1114.

Thereafter, the vertical or substantially vertical second surface 1112b2 of the protrusion part 1112b of the first contact part 1112 may meet and engage with the vertical or substantially vertical surface of the second tooth 1121p2 of the first driving gear 1121 according to the forward motion M1 of the first contact part 1112, and the force of the forward motion M1 through the first contact part 1112 may be transmitted so that the first driving gear 1121 is rotated by the second tooth 1121p2.

As the forward motion M1 and the backward motion M2 of the first contact part 1112 are sequentially performed multiple times, the first driving gear 1121 may be rotated only in one direction K1.

As the first driving gear 1121 is rotated in one direction K1, the transmission gear 1110n having the same shaft as the first driving gear 1121 may be first rotated in the same first direction N1. In addition, the transmission gears 1110n connected to the first driving gear 1121 may be rotated alternately along the first direction N1 and the second direction N2 opposite thereto.

Meanwhile, the transmission gears 1110n may be disposed to be misaligned with each other. For example, the first transmission gear 1110n1 and the second transmission gear 1110n2 may be alternately disposed to be misaligned with each other.

Accordingly, the first transmission gear 1110n1 may be disposed to be in sequential contact with and coupled to the first rack member 1143p1 of the rack member 1143p, and the second transmission gear 1110n2 may be disposed to be in sequential contact with and coupled to the second rack 1143p2 of the rack member 1143p.

As illustrated in FIG. 12, the transmission gears 1110n connected to the first driving gear 1121 may be rotated alternately along the first direction N1 and the second direction N2 opposite thereto through the rotational motion of the first driving gear 1121 in one direction K1. Specifically, the first transmission gear 1110n1 may be rotated in the first direction N1 and the second transmission gear 1110n2 may be rotated in the second direction N2.

Through such an arrangement, a force in the same direction as a whole may be transmitted to the rack member 1143p, and the rack member 1143p may perform a forward motion D1. Accordingly, the operation member 1140 may also be moved forward. For example, the operation member 1140 may be moved toward the distal end 1101d of the first jaw 1101.

The motion of the operation member 1140 is described with reference to FIGS. 14 and 15.

Referring to FIGS. 14 and 15, the reciprocating moving part 1110 may perform the forward motion M1 and the backward motion M2 as a whole. At this time, the reciprocating moving part 1110 is in the second position, that is, the position where the second contact part 1113 is in contact with the second driving gear 1122 of the driving gear 1120.

For example, as described above, the second position of the reciprocating moving part 1110 may be a position farther away from the operation member 1140 than the first position illustrated in FIGS. 8 and 12 described above, or may be a position disposed behind the first position.

In this case, the second driving gear 1122 may be rotated in one direction K2, for example, a counterclockwise direction, according to the forward motion M1 of the second contact part 1113. At this time, the first driving gear 1121 is rotated integrally with the second driving gear 1122 or rotated in at least the same direction to transmit a rotational force to the plurality of transmission gears 1110n according to the rotation direction of the second driving gear 1122.

Of course, in this case, the first contact part 1112 may be spaced apart from the first driving gear 1121 and the second driving gear 1122. Accordingly, the driving force through the first contact part 1112 is not transmitted to the first driving gear 1121.

Specifically, the second driving gear 1122 may have the form of a ratchet. For example, the second driving gear 1122 may include a plurality of teeth 1122p1 and 1122p2. As a specific example, the second driving gear 1122 may include the first teeth 1122p1 each having an inclined surface and a vertical or substantially vertical surface.

The second surface 1113b2 of the protrusion part 1113b of the second contact part 1113 may meet and engage with the vertical or substantially vertical surface of the first tooth 1122p1 of the second driving gear 1122, and the force of the forward motion M1 through the second contact part 1113 may be transmitted so that the second driving gear 1122 is rotated by the first tooth 1122p1.

During the backward motion M2 of the second contact part 1113, the protrusion part 1113b of the second contact part 1113 may correspond to the inclined surface of the second tooth 1122p2 of the second driving gear 1122, the protrusion part 1113b may slide over the inclined surface of the second tooth 1122p2, and the second driving gear 1122 may maintain a stationary state.

At this time, the second contact part 1113 may be rotated while being pushed outward and may come into close contact with the second driving gear 1122 again through the second elastic member 1115.

Thereafter, the protrusion part 1112b of the second contact part 1113 may meet and engage with the vertical or substantially vertical surface of the second tooth 1122p2 of the second driving gear 1122 according to the forward motion M1 of the second contact part 1113, and the force of the forward motion M1 through the second contact part 1112 may be transmitted so that the second driving gear 1122 is rotated by the second tooth 1122p2.

As the forward motion M1 and the backward motion M2 of the second contact part 1113 are sequentially performed multiple times, the second driving gear 1122 may be rotated only in one direction K2.

As the second driving gear 1122 is rotated in one direction K2, the transmission gear 1110n having the same shaft may be first rotated in the same second direction N2. In addition, the transmission gears 1110n connected to the second driving gear 1122 may be rotated alternately along the first direction N1 and the second direction N2 opposite thereto. At this time, the first driving gear 1121 may be moved integrally with or together with the second driving gear 1122. Due to such a structure, the driving force of the second driving gear 1122 may be transmitted to the transmission gear 1110n through the first driving gear 1121. In this case, since the first driving gear 1121 is spaced apart from the first contact part 1112, the driving force through the first contact part 1112 is not received.

Meanwhile, the transmission gears 1110n may be disposed to be misaligned with each other. For example, the first transmission gear 1110n1 and the second transmission gear 1110n2 may be alternately disposed to be misaligned with each other.

Accordingly, the first transmission gear 1110n1 may be disposed to be in sequential contact with and coupled to the first rack member 1143p1 of the rack member 1143p, and the second transmission gear 1110n2 may be disposed to be in sequential contact with and coupled to the second rack 1143p2 of the rack member 1143p.

As illustrated in FIG. 14, the transmission gears 1110n connected to the second driving gear 1122 may be rotated alternately along the first direction N1 and the second direction N2 opposite thereto through the rotational motion of the second driving gear 1122 in one direction K2. Specifically, the first transmission gear 1110n1 may be rotated in the second direction N2 and the second transmission gear 1110n2 may be rotated in the first direction N1.

Through such an arrangement, a force in the same direction as a whole may be transmitted to the rack member 1143p, and the rack member 1143p may perform a backward motion D2. Accordingly, the operation member 1140 may also perform a backward motion. For example, the operation member 1140 may be moved toward the proximal end 1101p of the first jaw 1101.

As a result, the linear motion of the reciprocating moving part 1110 causes the driving gear 1120 to rotate, and the rotational motion of the driving gear 1120 causes the transmission gear 1110n to rotate. The rotation of the transmission gear 1110n causes the rack member 1143p to rotate, and the rack member 1143p and the operation member 1140 may be moved together. The force transmitted through the driving gear 1120, the transmission gear 1110n, and the rack member 1143p may be transmitted to the operation member 1140, and thus, the motion of the operation member 1140 may be precisely controlled.

Meanwhile, although not illustrated, as another alternative embodiment, when the reciprocating moving part 1110 performs the forward motion and the backward motion at two points, the two points, which are a first position and a second position, may be spaced apart in a direction crossing or perpendicular to the reciprocating moving direction of the reciprocating moving part 1110, instead of being spaced apart in the reciprocating moving direction of the reciprocating moving part 1110. For example, the reciprocating moving part 1110 may have only one contact part. As a specific example, the reciprocating moving part 1110 may have only the first contact part 1112. The first contact part 1112 may be located at a lower position (a first position) and an upper position (a second position) spaced apart from the lower position. To this end, the reciprocating moving part 1110 may be configured to be moved upward and downward. At the first position, which is the lower position, the first contact part 1112 of the reciprocating moving part 1110 may be in contact with the first driving gear 1121 of the driving gear 1120. At the second position, which is the upper position higher than the lower position, the first contact part 1112 may be in contact with the second driving gear 1122 of the driving gear 1120. Due to such a structure, design convenience may be improved by reducing the space corresponding to the length for the motion of the reciprocating moving part 1110 in the end tool 1100 or the jaw 1103, and the jaw 1103 may be implemented compactly.

The cartridge 1550 and the stapling motion are described.

FIG. 16 is a perspective view illustrating the first jaw and the cartridge of the surgical instrument of FIG. 1. FIGS. 17 and 18 are cross-sectional views illustrating the overall stapling motion of the end tool of the surgical instrument of FIG. 1.

Referring to FIGS. 1 and 16, the cartridge 1500 may be disposed in the first jaw 1101. For example, the cartridge 1500 may be coupled to and disposed in the cartridge accommodation part 1101a of the first jaw 1101. For example, the cartridge 1500 may be integrally formed with the first jaw 1101 while the operation member 1140 is disposed in the first jaw 1101. In addition, as an alternative embodiment, the cartridge 1500 may be formed to be attachable to and detachable from the first jaw 1101.

The cartridge 1500 may include a plurality of staples 1530 therein to suture tissue and may perform cutting through the operation member 1140. Here, the cartridge 1500 may include a cover 1510, staples 1530, and a withdrawal member 1535.

The cartridge accommodation part 1101a of the first jaw 1101 may be configured to accommodate the operation member 1140 and the staple 1530. The cartridge accommodation part 1101a may have a bottom and a side surface in the form of a concave groove in cross section and may be formed in an approximately “U” shape.

The cover 1510 may be configured to cover the upper portion of the cartridge accommodation part 1101a of the first jaw 1101. Staple holes 1510s through which the staples 1530 may be ejected to the outside may be formed in the cover 1510. As the staples 1530, which are accommodated inside the cartridge accommodation part 1101a before a stapling motion, are pushed and raised upward by the operation member 1140 during a stapling motion and pass through the staple holes 1510s of the cover 1510 to be withdrawn to the outside of the cartridge 1500, stapling is performed.

Meanwhile, a slit 1510G may be formed in the cover 1510 along a length direction of the cover 1510. A blade 1142 of the operation member 1140 may protrude out of the cartridge 1500 through the slit 1510G. As the blade 1142 of the operation member 1140 passes along the slit 1510G, staple-completed tissue may be cut.

The staples 1530 may be disposed inside the cartridge accommodation part 1101a of the first jaw 1101. As the operation member 1140 is linearly moved in one direction, the staples 1530 are sequentially pushed and raised from the inside of the cartridge accommodation part 1101a to the outside, thereby performing suturing, that is, stapling. The staples 1530 may be made of a material that is durable and does not have an abnormal effect on the human body. For example, the staples 1530 may be made of a material that may include titanium, stainless steel, or the like.

Meanwhile, the withdrawal member 1535 may be further disposed between the cartridge accommodation part 1101a and the staple 530. In other words, it may be said that the staple 1530 is disposed above the withdrawal member 1535. In this case, the operation member 1540 is linearly moved in one direction to push and raise the withdrawal member 1535, and the withdrawal member 1535 may push and raise the staples 1530.

As such, in both the case where the operation member 1140 directly pushes and raises the staples 1530 and the case where the operation member 1140 pushes and raises the withdrawal member 1535 so that the withdrawal member 1535 pushes and raises the staples 1530 (i.e., a case where the operation member 1140 indirectly pushes and raises the staples 1530), it may be said that the operation member 1140 pushes and raises the staples 1530.

As described above, the operation member 1140 may be disposed inside the cartridge accommodation part 1101a of the first jaw 1101. The operation member 1140 may be moved along the longitudinal direction of the first jaw 1101 while corresponding to the first guide groove 1101h of the first jaw 1101.

In addition, the operation member 1140 may include the wedge WDG (see FIGS. 7, 17, and 18) or may be used together with the wedge. When the operation member 1140 is moved, the wedge may be moved together the operation member 1140 so that the wedge may directly push and raise the staple 1530, or the wedge may push and raise the withdrawal member 1535 so that the withdrawal member 1535 pushes and raises the staple 1530.

The clamp 1146 may be formed on one side of the blade 1142 and may be formed in the form substantially parallel to the main body 1545. In addition, the clamp 1146 may pass through the groove of the second jaw 1102 and come into contact with the upper surface of the second jaw 1102, and as the operation member 1140 is moved, a force may be applied in close contact with the second jaw 1102 in a direction in which the second jaw 1102 approaches the first jaw 1101.

As described above, the motion of the reciprocating moving part 1110, for example, the linear motion, causes the first contact part 1112 and the second contact part 1113 to rotate the driving gear 1120. Due to the rotational force, the transmission gears 1110n are sequentially rotated. Accordingly, the rack member 1143p coupled to the transmission gear 1110n may be moved and the operation member 1140 may be moved integrally with the rack member 1143p. For example, the operation member may perform a forward motion toward the distal end 1101d of the first jaw 1101.

The wedge may push and raise the withdrawal member 1535 through the forward motion of the operation member 1140, and thus, the staple 1530 may also be raised. In addition, cutting may be performed through the blade 1142 of the operation member 1140.

Referring to FIGS. 17 and 18, in the state as shown in FIG. 17A, as the operation member 1140 is moved in the direction of an arrow A1 of FIG. 17B, the wedge, specifically the inclined surface WDG1 of the wedge, pushes and raises the withdrawal member 1535, and the withdrawal member 1535 pushes and raises one side of a lower portion of the staple 1530. In addition, due thereto, the staple 1530 is ejected to the outside of the first jaw 1101 and the cartridge 1500.

In this state, when the operation member 1140 is further moved in the direction of an arrow A2 of FIG. 17C, the ejected staple 1530 is continuously pushed and raised by the operation member 1540 while in contact with a lower surface of the second jaw 1102, for example, an anvil of the second jaw 1102, so that stapling is performed while both end portions of the staple 1530 are bent.

As such motions are continuously performed, stapling is sequentially performed from the staple 1530 at the proximal end (1101p of FIG. 3) side to the staple 1530 at the distal end (1101d of FIG. 3) side among the plurality of staples 1530, as shown in FIG. 18.

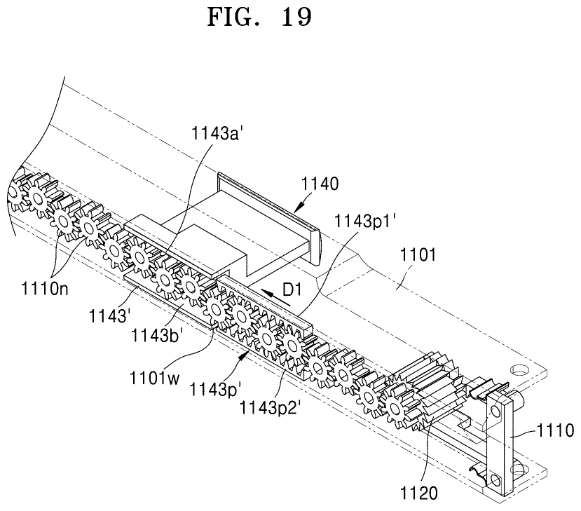

FIG. 19 is a diagram illustrating a modification of the end tool of FIG. 2.

Referring to FIG. 19, the present modification differs from the embodiments described above in terms of an operation member 1140′ and a rack member 1143p′, and thus, such differences are described below.

The rack member 1143p′ of the present modification may be formed to have a plurality of concavo-convex portions.

The rack member 1143p′ may be disposed separately from the operation member 1140′. That is, the rack member 1143p′ is not formed on a side portion 1143′ of the operation member 1140′.

Specifically, the rack member 1143p′ may be disposed behind the operation member 1140′. For example, the rack member 1143p′ may be disposed closer to the proximal end (1101p of FIG. 3) of the first jaw than the operation member 1140′. It may also be said that the rack member 1143p′ may be disposed closer to the reciprocating moving part 1110 than the operation member 1140′. It may also be said that the rack member 1143p′ may be disposed between the operation member 1140′ and the reciprocating moving part 1110.

The rack member 1143p′ may have a plurality of members. As a specific example, the rack member 1143p′ may include a first rack member 1143p1′ and a second rack member 1143p2′.

The first rack member 1143p1′ and the second rack member 1143p2′ may be disposed to be spaced apart from each other and face each other, and the concavo-convex portion of the first rack member 1143p1′ and the concavo-convex portion of the second rack member 1143p2′ may be disposed to face each other.

For example, the first rack member 1143p1′ and the second rack member 1143p2′ may each be configured to be movable while being disposed in the extension groove 1101w of the first jaw.

The width of the rack member 1143p′ may be equal to the width of the operation member 1140′ so that the rack member 1143p′ is in contact with the operation member 1140′ and is movable while pushing in one direction, or the shape and offset of the first rack member 1143p1′ and the second rack member 1143p2′ may be determined so that at least a region overlapping a first side portion 1143a′ and a second side portion 1143b′ of the side portion 1143′ of the operation member 1140′ is formed.

Similar to the embodiments described above, the driving gear 1120 is rotated through the motion of the reciprocating moving part 1110, and the plurality of transmission gears 1110n are also rotated according to the rotation of the driving gear 1120. Due to the rotational motion of the transmission gear 1110n, the rack member 1143p′ may perform a motion in one direction D1, for example, a forward motion, while in contact with and coupled to the transmission gear 1110n.

When the rack member 1143p′ continues to perform a forward motion in one direction D1 while being spaced apart from the operation member 1140′, the rack member 1143p′ comes into contact with the operation member 1140′. When the rack member 1143p′ is further moved forward in one direction D1, the rack member 1143p′ pushes the operation member 1140′ in one direction D1. As a result, the operation member 1140′ may also perform a motion in one direction S1, for example, a forward motion.

That is, in the present modification, the rack member 1143p′ is provided separately from the operation member 1140′, so as to facilitate the mutual manufacture of the rack member 1143p′ and the operation member 1140′ and improve the efficiency of placement and installation in the first jaw 1101.

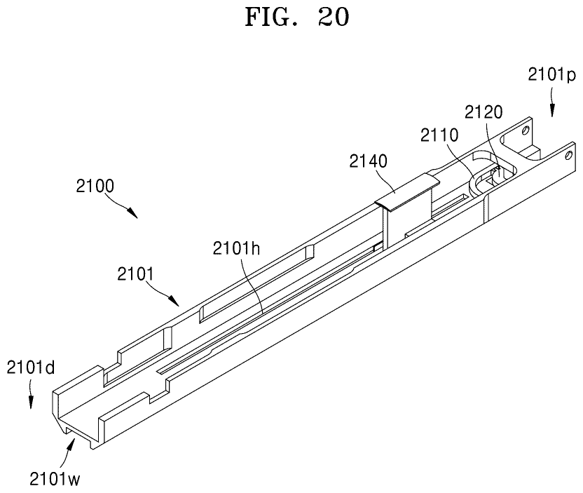

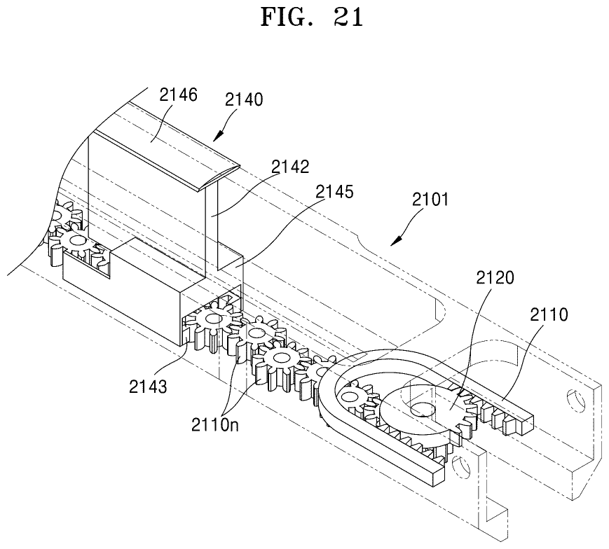

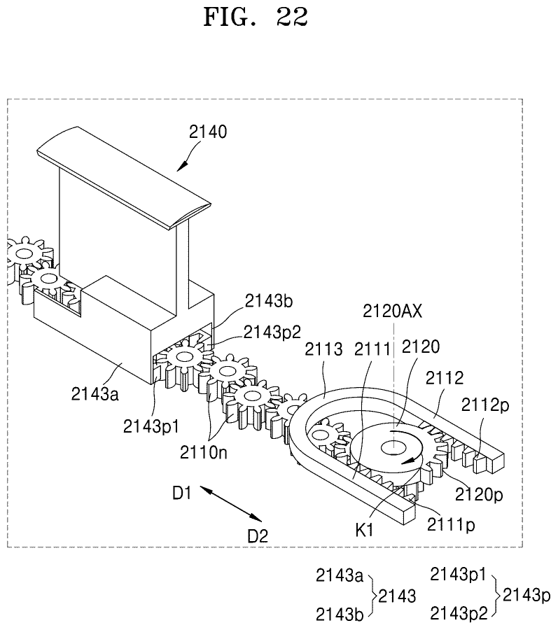

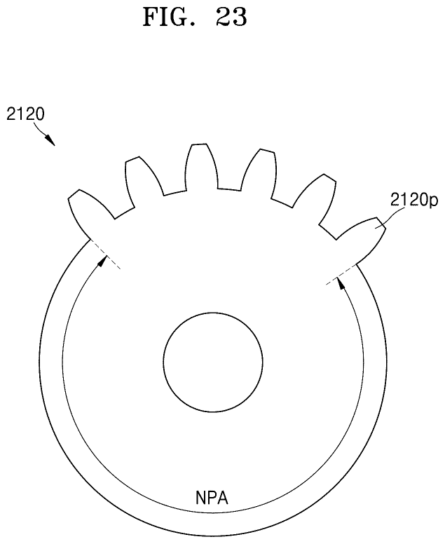

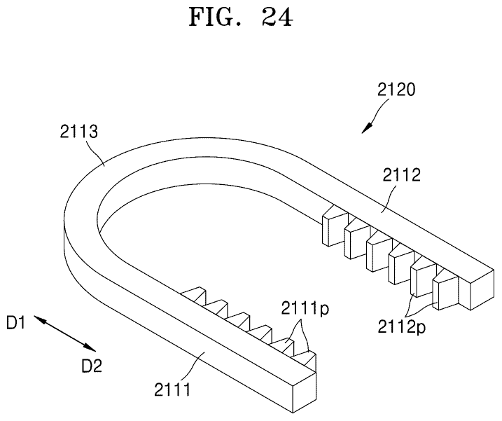

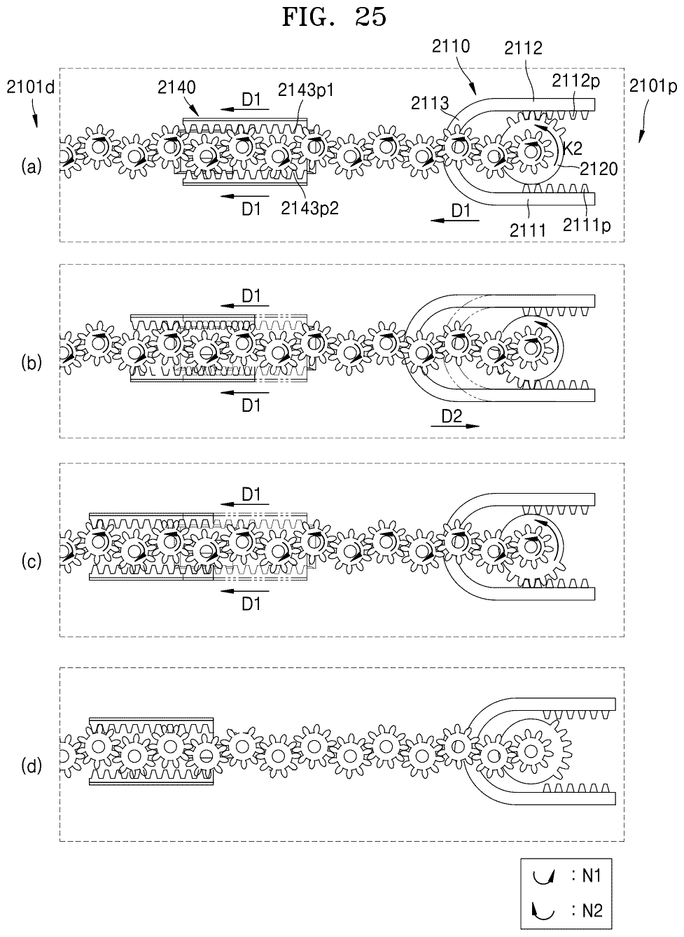

FIG. 20 is a schematic perspective view for describing an end tool of a surgical instrument according to another embodiment. FIGS. 21 and 22 are schematic perspective views for describing an operation member and a reciprocating moving part of the end tool of FIG. 20. FIG. 23 is a plan view illustrating a driving gear of the end tool of FIG. 20. FIG. 24 is a perspective view illustrating the reciprocating moving part of the end tool of FIG. 20. FIG. 25 is a diagram for describing the operation of the operation member of the end tool of FIG. 20.

An end tool 2100 of the present embodiment may include a jaw, a reciprocating moving part 2110, and a plurality of transmission gears 2110n. For convenience of explanation, the description will focus on differences from the embodiments described above. The present embodiment differs from the embodiments described above in terms of the reciprocating moving part 2110 and a driving gear 2120, and thus, the description will focus on such differences.

Referring to FIG. 20, the jaw of the end tool 2100 is illustrated. For convenience of explanation, a first jaw 2101 is mainly illustrated, and a second jaw, a cartridge, and the like are omitted.

The first jaw 2101 may be formed in the shape of an elongated bar as a whole, a cartridge (1500 of FIG. 1) may be accommodated in the first jaw 2101 at a distal end 2101d side, and a rotation shaft may be disposed at a proximal end 2101p so that the first jaw 2101 is rotatable around the rotation shaft. The first jaw 2101 may be formed entirely in the form of a hollow box, of which one surface (upper surface) is removed, so that a cartridge accommodation part 2101a capable of accommodating the cartridge may be formed inside the first jaw 2101. That is, the first jaw 2101 may be formed in an approximately “U” shape in cross section.

One or more first guide grooves 2101h may be formed on the bottom surface of the first jaw 2101, that is, on the bottom surface facing the upper open region in which one surface is removed. The first guide groove 2101h may have a plurality of grooves. Since this is substantially the same as the first jaw 1101 of the embodiment described above, a detailed description thereof is omitted.

The first jaw 2101 may include an extension groove 2101w in a region facing the back surface thereof. Since this is substantially the same as the first jaw 1101 of the embodiment described above, a detailed description thereof is omitted.

A plurality of rotation shafts may be formed to protrude from one surface of the extension groove 2101w. Since this is substantially the same as the rotation shaft 1101c of the first jaw 1101 described above, a detailed description thereof is omitted.

The operation member 2140 may include a main body 2145, a blade 2142, and a side portion 2143. Meanwhile, the operation member 2140 may be used together with the wedge WDG (see FIGS. 7, 17, and 18).

As an alternative embodiment, a clamp 2146 may be further formed in one region of the blade 2142. The side portion 2143 may be formed to have a height in a region of the main body 2145 in a direction opposite to the protruding direction of the blade 2142, and may include a first side portion 2143a and a second side portion 2143b that protrude from both sides in the width direction of the main body 2145.

A rack member 2143p having a plurality of concavo-convex portions may be formed on the side portion 2143 and may be formed on the first side portion 2143a and the second side portion 2143b.

For example, the rack member 2143p may have a plurality of members. As a specific example, the rack member 2143p may include a first rack member 2143p1 and a second rack member 2143p2. As the rack member 2143p is moved, the rack member 2143p and the operation member 2140 may be moved integrally with each other.

Since the operation member 2140 and the rack member 2143p are substantially the same as the operation member 2140 and the rack member 2143p of the embodiment described above, a detailed description thereof is omitted.

The reciprocating moving part 2110, the driving gear 2120, and the like are described in more detail.

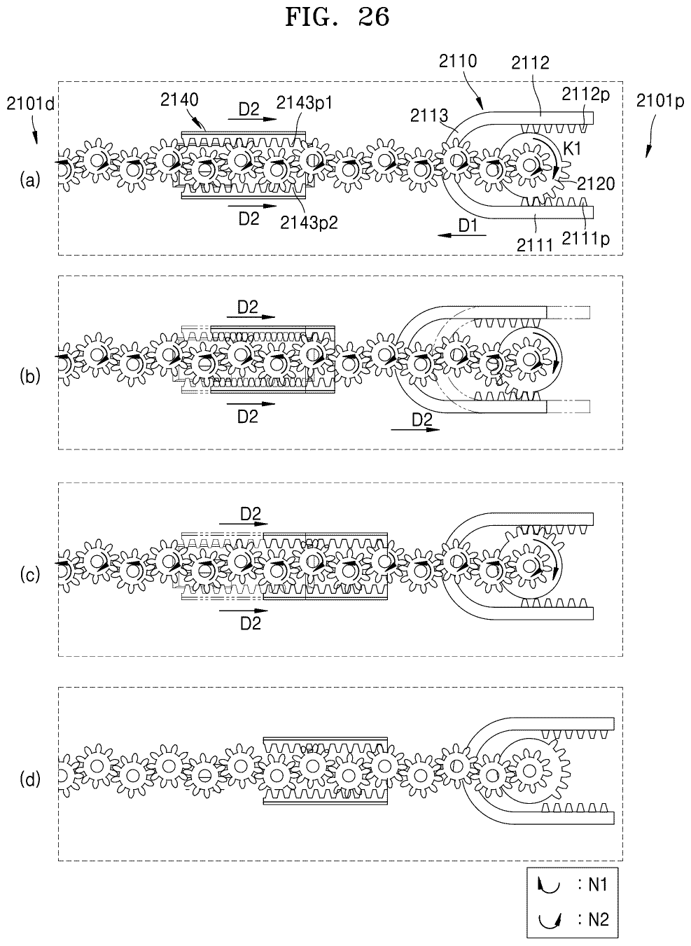

FIGS. 21 and 22 are schematic perspective views for describing the operation member and the reciprocating moving part of the end tool of FIG. 20. FIG. 23 is a plan view illustrating the driving gear of the end tool of FIG. 20. FIG. 24 is a perspective view illustrating the reciprocating moving part of the end tool of FIG. 20. FIGS. 25 and 26 are diagrams for describing the operation of the operation member of the end tool of FIG. 20.

Referring to FIGS. 21 and 22, the reciprocating moving part 2110 may be configured to perform a reciprocating linear motion in both directions toward the proximal end 2101p and the distal end 2101d of the first jaw 2101. As a specific example, as illustrated in FIG. 22, the reciprocating moving part 2110 may be configured to perform a reciprocating motion of a forward motion D1 and a backward motion D2.

When the reciprocating moving part 2110 is linearly moved, the driving gear 2120 connected thereto may be moved. For example, the driving gear 2120 may be rotated in one direction (K1 of FIG. 22).

A plurality of transmission gears 2110n may be rotated according to the rotational motion of the driving gear 2120. The rack member 2143p may be moved due to such a rotational motion, and the operation member 2140 may be moved together with the rack member 2143p.

Referring to the embodiment of FIG. 9 described above, the transmission gear 2110n has a circular shape and may have a groove corresponding to the rotation shaft (1101c of FIG. 4) at the center thereof, and a plurality of teeth may be formed on the outer circumferential surface thereof.

Referring to FIGS. 21, 22, and 24, the reciprocating moving part 2110 may include a main body 2113, a first rack gear part 2111, and a second rack gear part 2112. The main body 2113 may be located ahead of the first rack gear part 2111 and the second rack gear part 2112, that is, close to the operation member 2140.

The main body 2113 may have a curved portion. For example, the main body 2113 may have a curved surface corresponding to the curved shape of the driving gear 2120. For example, the main body 2113 may have a curved surface that is convex toward the operation member 2140.

The first rack gear part 2111 and the second rack gear part 2112 may be configured to be connected to the main body 2113 while being spaced apart from each other. The first rack gear part 2111 and the second rack gear part 2112 may each be formed to have a length from the main body 2113 along the longitudinal direction of the first jaw 2101 and may extend in a direction away from the operation member 2140.

The first rack gear part 2111 and the second rack gear part 2112 may be formed to have the same length and may be formed parallel to each other and may be offset and spaced apart from each other.

The first rack gear part 2111 may have the form of a concave-convex portion having a plurality of irregularities 2111p. The irregularities 2111p may be formed to correspond to the teeth 2120p of the driving gear 2120.

Accordingly, when the first rack gear part 2111 performs a linear motion D1 and D2, the irregularities 2111p of the first rack gear part 2111 and the teeth 2120p of the driving gear 2120 may be engaged with each other so that the driving gear 2120 may be rotated according to the linear motion of the first rack gear part 2111. As the driving gear 2120 is rotated, the plurality of transmission gears 2110n connected thereto may be rotated. As a result, the rack member 2143p and the operation member 2140 may be moved. Details thereof are described later.

The second rack gear part 2112 may have the form of a concave-convex portion having a plurality of irregularities 2112p. The irregularities 2112p may be formed to correspond to the teeth 2120p of the driving gear 2120.

Accordingly, when the second rack gear part 2112 performs a linear motion D1 and D2, the irregularities 2112p of the second rack gear part 2112 and the teeth 2120p of the driving gear 2120 may be engaged with each other so that the driving gear 2120 may be rotated according to the linear motion of the second rack gear part 2112. As the driving gear 2120 is rotated, the plurality of transmission gears 2110n connected thereto may be rotated. As a result, the rack member 2143p and the operation member 2140 may be moved. Details thereof are described later.

Referring to FIGS. 22, 23, and the like, the driving gear 2120 is configured to be rotatable around a rotation shaft 2120AX. The driving gear 2120 may have the form of a concave-convex portion having a plurality of teeth 2120p.

The driving gear 2120 may have a non-formation area NPA formed on the outer circumferential surface so as to be adjacent to an area where the plurality of teeth 2120p are formed. The non-formation area NPA is an area where none of the plurality of teeth 2120p are not formed. For example, the non-formation area NPA may be formed to partially correspond to a circle or an arc.

The driving of the reciprocating moving part 2110 and the resulting motion of the operation member 2140 are described.

FIG. 22 is a schematic perspective view for describing the operation member and the reciprocating moving part of the end tool of FIG. 20, and the first jaw is omitted for convenience of explanation.

FIGS. 25 and 26 are diagrams for describing the operation of the operation member of the end tool of FIG. 20. For convenience of explanation, the first jaw is omitted. Specifically, FIGS. 25 and 26 may be diagrams viewed from below the first jaw, that is, from the back surface thereof. In addition, as a specific example, FIG. 25 may be a diagram for describing the forward motion of the operation member and FIG. 26 may be a diagram for describing the backward motion of the operation member.

The reciprocating moving part 1110 may be formed to receive a driving force from a driving part and perform a linear motion, for example, a forward motion M1 and a backward motion M2 in FIG. 22. As a specific example, rotation of a motor part in one direction may be converted into a bidirectional linear motion through connection of various members, such as pulleys and links, and the bidirectional linear motion may be then transmitted to the reciprocating moving part 2110. In addition, the linear motion of the driving part may be transmitted to the reciprocating moving part 2110 in various other methods. For example, the linear motion of the driving part may be directly transmitted to the reciprocating moving part 2110.

Referring to FIGS. 22 and 25, the reciprocating moving part 2110 may perform the forward motion M1 and the backward motion M2 as a whole. At this time, the irregularities 2111p of the first rack gear part 2111 and the irregularities 2112p of the second rack gear part 2112 in the reciprocating moving part 2110 are moved in engagement with the teeth 2120p of the driving gear 2120. Accordingly, the driving gear 2120 may be rotated in one direction K2.

As a specific example, when the reciprocating moving part 2110 is moved forward in the direction D1 of FIG. 25A, the second rack gear part 2112 of the reciprocating moving part 2110 and the teeth 2120p of the driving gear 2120 are moved in engagement with each other, and accordingly, the driving gear 2120 may be rotated in one direction K2, for example, a counterclockwise direction. At this time, the first rack gear part 2111 of the reciprocating moving part 2110 may not correspond to the teeth 2120p of the driving gear 2120, and may correspond to the non-formation area NPA.

As the driving gear 2120 is rotated in one direction K2, the transmission gear 2110n having the same shaft as the driving gear 2120 may be first rotated in the same second direction N2. That is, the plurality of transmission gears 2110n from the driving gear 2120 side may be rotated alternately along the second direction N2 and the first direction N1 opposite thereto.

Meanwhile, the plurality of transmission gears 2110n may be disposed to be misaligned with each other. For example, the first transmission gear 2110n1 and the second transmission gear 2110n2 may be alternately disposed to be misaligned with each other.

Accordingly, the first transmission gear 2110n1 may be disposed to be in sequential contact with and coupled to the first rack member 2143p1 of the rack member 2143p, and the second transmission gear 2110n2 may be disposed to be in sequential contact with and coupled to the second rack 2143p2 of the rack member 2143p.

Accordingly, the plurality of transmission gears 2110n connected to the driving gear 2120 may be rotated alternately along the second direction N2 and the first direction N1 opposite thereto through the rotational motion of the driving gear 2120 in one direction K2. Specifically, the first transmission gear 2110n1 may be rotated in the second direction N2 and the second transmission gear 2110n2 may be rotated in the first direction N1. A force in the same direction as a whole may be transmitted to the rack member 2143p, and the rack member 2143p may perform a forward motion D1. Therefore, the operation member 2140 may also be moved forward. For example, the operation member 2140 may be moved toward the distal end 2101d of the first jaw 2101. Referring to the drawings, the reciprocating moving part 2110 is in a forward-moved position as illustrated in FIG. 25B, and the operation member 2140 is also in a position after the forward motion D1.

Thereafter, when the reciprocating moving part 2110 is moved backward, that is, when the reciprocating moving part 2110 is moved in the D2 direction of FIG. 25B, the first rack gear part 2111 of the reciprocating moving part 2110 and the teeth 2120p of the driving gear 2120 are moved in engagement with each other, and accordingly, the driving gear 2120 may be rotated in one direction K2, for example, a counterclockwise direction. At this time, the second rack gear part 2112 of the reciprocating moving part 2110 may not correspond to the teeth 2120p of the driving gear 2120, and may correspond to the non-formation area NPA.

As the driving gear 2120 is rotated in one direction K2, the transmission gear 2110n having the same shaft as the driving gear 2120 may be first rotated in the same second direction N2. In addition, the plurality of transmission gears 2110n including this and connected thereto may be rotated alternately along the second direction N2 and the first direction N1 opposite thereto.

Meanwhile, the plurality of transmission gears 2110n may be disposed to be misaligned with each other. For example, the first transmission gear 2110n1 and the second transmission gear 2110n2 may be alternately disposed to be misaligned with each other.

Accordingly, the first transmission gear 2110n1 may be disposed to be in sequential contact with and coupled to the first rack member 2143p1 of the rack member 2143p, and the second transmission gear 2110n2 may be disposed to be in sequential contact with and coupled to the second rack 2143p2 of the rack member 2143p.

Accordingly, the plurality of transmission gears 2110n connected to the driving gear 2120 may be rotated alternately along the second direction N2 and the first direction N1 opposite thereto through the rotational motion of the driving gear 2120 in one direction K2. Specifically, the first transmission gear 2110n1 may be rotated in the second direction N2 and the second transmission gear 2110n2 may be rotated in the first direction N1. A force in the same direction as a whole may be transmitted to the rack member 2143p, and the rack member 2143p may perform a forward motion D1. Therefore, the operation member 2140 may also be moved forward. For example, the operation member 2140 may be moved toward the distal end 2101d of the first jaw 2101. Accordingly, the reciprocating moving part 2110 is in a backward-moved position as illustrated in FIG. 25C, and the operation member 2140 is also in a position after the further forward motion D1.

As described with reference to FIGS. 22 and 25, the driving gear 2120 may be rotated in one direction during the forward motion D1 and the backward motion D2 of the reciprocating moving part 2110. That is, the driving gear 2120 may be rotated in one direction K2 of the counterclockwise direction by the second rack gear part 2112 while the reciprocating moving part 2110 is moved in one direction (for example, during the forward motion D1), and the driving gear 2120 may also be rotated in one direction K2 of the counterclockwise direction by the first rack gear part 2111 while the reciprocating moving part 2110 is moved in another direction (for example, during the backward motion D2). Accordingly, the operation member 2140 is moved forward during the forward motion D1 and the backward motion D2 of the reciprocating moving part 2110, thereby improving the operation efficiency and the movement of the operation member 2140.

Meanwhile, as illustrated in FIG. 25D, when the reciprocating moving part 2110 performs the motions D1 and D2, for example, when the reciprocating moving part 2110 repeats the forward motion and the backward motion, the teeth 2120p of the driving gear 2120 may not correspond to or engage with the irregularities 2111p of the first rack gear part 2111 and the irregularities 2112p of the second rack gear part 2112 at one or more time points. For example, the non-formation area NPA of the driving gear 2120 may correspond to the irregularities 2111p of the first rack gear part 2111 and the irregularities 2112p of the second rack gear part 2112.

In this case, the driving force of the motions D1 and D2 of the reciprocating moving part 2110 may not be transmitted to the driving gear 2120, and this time point may be referred to as a dead zone. A modification that compensates for the dead zone will be described later. Although not illustrated, unlike in FIG. 25D, there may be a dead zone in which the teeth 2120p of the driving gear 2120 face a left side.

Meanwhile, referring to FIGS. 22 and 26, the operation member 2140 may be moved backward. That is, the operation member 2140 may be moved toward the proximal end. As a specific example, when the reciprocating moving part 2110 is moved forward in the direction D1 of FIG. 26A, the first rack gear part 2111 of the reciprocating moving part 2110 and the teeth 2120p of the driving gear 2120 are moved in engagement with each other, and accordingly, the driving gear 2120 may be rotated in one direction K1, for example, a clockwise direction. At this time, the second rack gear part 2112 of the reciprocating moving part 2110 may not correspond to the teeth 2120p of the driving gear 2120, and may correspond to the non-formation area NPA.

As the driving gear 2120 is rotated in one direction K1, the transmission gear 2110n having the same shaft as the driving gear 2120 may be first rotated in the same first direction N1. That is, the plurality of transmission gears 2110n from the driving gear 2120 side may be rotated alternately along the first direction N1 and the second direction N2 opposite thereto.

Meanwhile, the plurality of transmission gears 2110n may be disposed to be misaligned with each other. For example, the first transmission gear 2110n1 and the second transmission gear 2110n2 may be alternately disposed to be misaligned with each other.

Accordingly, the first transmission gear 2110n1 may be disposed to be in sequential contact with and coupled to the first rack member 2143p1 of the rack member 2143p, and the second transmission gear 2110n2 may be disposed to be in sequential contact with and coupled to the second rack 2143p2 of the rack member 2143p.

Accordingly, the plurality of transmission gears 2110n connected to the driving gear 2120 may be rotated alternately along the first direction N1 and the second direction N2 opposite thereto through the rotational motion of the driving gear 2120 in one direction K1. Specifically, the first transmission gear 2110n1 may be rotated in the first direction N1 and the second transmission gear 2110n2 may be rotated in the second direction N2. A force in the same direction as a whole may be transmitted to the rack member 2143p, and the rack member 2143p may perform a backward motion D2. Therefore, the operation member 2140 may also be moved backward. For example, the operation member 2140 may be moved toward the proximal end 2101p of the first jaw 2101. Referring to the drawings, the reciprocating moving part 2110 is in a forward-moved position as illustrated in FIG. 26B, and the operation member 2140 is also in a position after the backward motion D2.

Thereafter, when the reciprocating moving part 2110 is moved backward, that is, when the reciprocating moving part 2110 is moved in the D2 direction of FIG. 26B, the second rack gear part 2112 of the reciprocating moving part 2110 and the teeth 2120p of the driving gear 2120 are moved in engagement with each other, and accordingly, the driving gear 2120 may be rotated in one direction K1, for example, a clockwise direction. At this time, the first rack gear part 2111 of the reciprocating moving part 2110 may not correspond to the teeth 2120p of the driving gear 2120, and may correspond to the non-formation area NPA.

As the driving gear 2120 is rotated in one direction K1, the transmission gear 2110n having the same shaft as the driving gear 2120 may be first rotated in the same first direction N1. In addition, the plurality of transmission gears 2110n including this and connected thereto may be rotated alternately along the first direction N1 and the second direction N2 opposite thereto.

Meanwhile, the plurality of transmission gears 2110n may be disposed to be misaligned with each other. For example, the first transmission gear 2110n1 and the second transmission gear 2110n2 may be alternately disposed to be misaligned with each other.

Accordingly, the first transmission gear 2110n1 may be disposed to be in sequential contact with and coupled to the first rack member 2143p1 of the rack member 2143p, and the second transmission gear 2110n2 may be disposed to be in sequential contact with and coupled to the second rack 2143p2 of the rack member 2143p.

Accordingly, the plurality of transmission gears 2110n connected to the driving gear 2120 may be rotated alternately along the first direction N1 and the second direction N2 opposite thereto through the rotational motion of the driving gear 2120 in one direction K1. Specifically, the first transmission gear 2110n1 may be rotated in the first direction N1 and the second transmission gear 2110n2 may be rotated in the second direction N2. A force in the same direction as a whole may be transmitted to the rack member 2143p, and the rack member 2143p may perform a backward motion D2. Therefore, the operation member 2140 may also be moved backward. For example, the operation member 2140 may be moved toward the proximal end 2101p of the first jaw 2101. Referring to the drawings, the reciprocating moving part 2110 is in a backward-moved position as illustrated in FIG. 26C, and the operation member 2140 is also in a position after the further backward motion D2 compared to FIG. 26B.

As described with reference to FIGS. 22 and 26, the driving gear 2120 may be rotated in one direction during the forward motion D1 and the backward motion D2 of the reciprocating moving part 2110. That is, the driving gear 2120 may be rotated in one direction K1 of the clockwise direction by the first rack gear part 2111 while the reciprocating moving part 2110 is moved in one direction (for example, during the forward motion D1), and the driving gear 2120 may also be rotated in one direction K1 of the clockwise direction by the second rack gear part 2112 while the reciprocating moving part 2110 is moved in another direction (for example, during the backward motion D2). Accordingly, the operation member 2140 is moved backward during the forward motion D1 and the backward motion D2 of the reciprocating moving part 2110, thereby improving the operation efficiency and the movement of the operation member 2140.

Meanwhile, as illustrated in FIG. 26D, when the reciprocating moving part 2110 performs the motions D1 and D2, for example, when the reciprocating moving part 2110 repeats the forward motion and the backward motion, the teeth 2120p of the driving gear 2120 may not correspond to or engage with the irregularities 2111p of the first rack gear part 2111 and the irregularities 2112p of the second rack gear part 2112 at one or more time points. For example, the non-formation area NPA of the driving gear 2120 may correspond to the irregularities 2111p of the first rack gear part 2111 and the irregularities 2112p of the second rack gear part 2112.

The driving force of the motions D1 and D2 of the reciprocating moving part 2110 may not be transmitted to the driving gear 2120, and this time point may be referred to as a dead zone. In addition, although not illustrated, unlike in FIG. 26D, there may be a dead zone in which the teeth 2120p of the driving gear 2120 face a right side.

A modification that compensates for the dead zone is described.

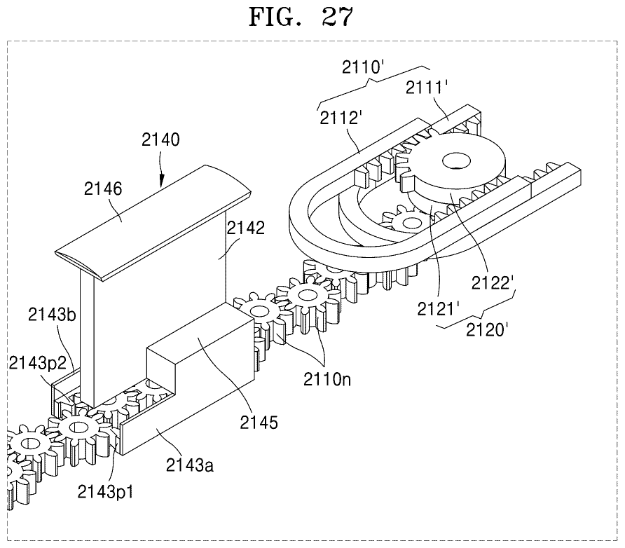

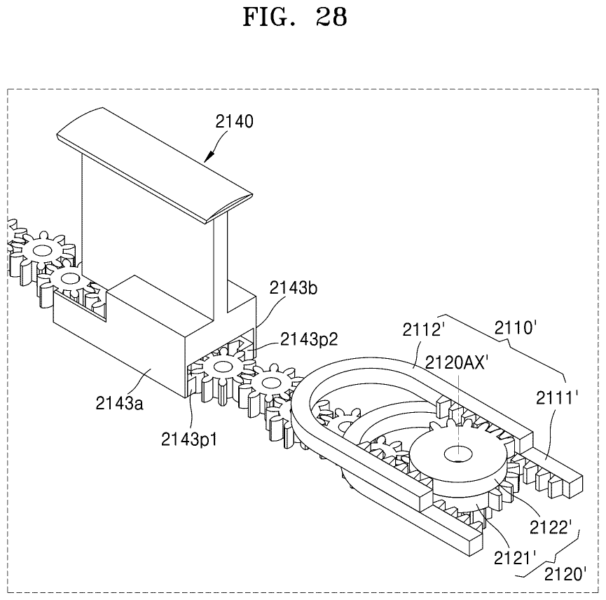

FIGS. 27 and 28 are diagrams illustrating a modification of the end tool of FIG. 20.

Referring to FIGS. 27 and 28, the present modification differs from the embodiments described above in terms of a reciprocating moving part 2110′ and a driving gear 2120′, and thus, such differences are described below.

The reciprocating moving part 2110′ of the present modification may have a plurality of moving parts. For example, the reciprocating moving part 2110′ of the present modification may include a first moving part 2111′ and a second moving part 2112′. Each of the first moving part 2111′ and the second moving part 2112′ includes a main body, a first rack gear part, and a second rack gear part. Each of the first moving part 2111′ and the second moving part 2112′ may have substantially the same shape as the reciprocating moving part 2110 of the embodiment described above.

The first moving part 2111′ and the second moving part 2112′ may be disposed up and down, that is, the first moving part 2111′ and the second moving part 2112′ may be disposed in a stacked form from below with respect to the bottom surface of the first jaw 2101.

The first moving part 2111′ and the second moving part 2112′ may be disposed to be misaligned with each other when disposed up and down. For example, the second moving part 2112′ may be in a forward position, compared to the first moving part 2111′. For example, the second moving part 2112′ may be in a position closer to the operation member 2140. Even when the first moving part 2111′ and the second moving part 2112′ are moved forward and backward integrally with each other, the time point or the time at which the first rack gear part and the second rack gear part of the first moving part 2111′ and the second moving part 2112′ correspond to the driving gear 2120′ may be set to be different. Accordingly, the driving efficiency may be improved by ensuring that the dead zone of the first driving gear 2121′ and the dead zone of the second driving gear 2122′ do not overlap each other in view of time.

In addition, it may be said that when the first driving gear 2121′ is in the dead zone, the time point or position at which the first moving part 2111′ corresponds to the first driving gear 2121′ is different from the time point or position at which the second moving part 2112′ corresponds to the second driving gear 2122′, the second driving gear 2122′ is not in the dead zone, and thus, may not receive power from the second moving part 2112′. In addition, since the first driving gear 2121′ and the second driving gear 2122′ are constrained to be rotated together, the second moving part 2112′ may use a force to rotate the second driving gear 2122′ and the first driving gear 2121′ may effectively escape the dead zone.

As an alternative embodiment, the first moving part 2111′ and the second moving part 2112′ may be disposed to be moved forward and backward. For example, the first moving part 2111′ and the second moving part 2112′ are not moved forward and backward integrally with each other at the same time, but are moved forward and backward at different times. As a specific example, the first moving part 2111′ and the second moving part 2112′ may be sequentially moved forward and backward.

As a more specific example, the time point at which the first moving part 2111′ and the second moving part 2112′ do not coincide with each other and are sequentially moved is described. For example, the first moving part 2111′ and the second moving part 2112′ may perform a reciprocating motion with a period T. At this time, T is a time unit (for example, seconds) and may be a time for which the first moving part 2111′ or the second moving part 2112′ performs one reciprocating motion of the forward and backward motions.

In this case, the time point at which the first moving part 2111′ completes the forward motion and starts the backward motion may have an offset from the time point at which the second moving part 2112′ completes the forward motion and starts the backward motion. As a specific example, the offset between the two time points may have a value different from 0.5 T. For example, the offset between the two time points may have a value less than or greater than 0.5 T. Accordingly, it is possible to limit the time point at which both the first moving part 2111′ and the second moving part 2112′ are in the dead zone.

The driving gear 2120′ of the present modification may have a plurality of driving gears. For example, the driving gear 2120′ may have a first driving gear 2121′ and a second driving gear 2122′. The first driving gear 2121′ and the second driving gear 2122′ may be rotatable around the same central shaft 2120AX′.

Each of the first driving gear 2121′ and the second driving gear 2122′ may have the form of a concave-convex portion having a plurality of teeth, and may also include a non-formation area. As a specific example, the first driving gear 2121′ and the second driving gear 2122′ may be substantially the same as the driving gear 2120 described above.

The first driving gear 2121′ and the second driving gear 2122′ may be disposed up and down, that is, the first driving gear 2121′ and the second driving gear 2122′ may be disposed in a stacked form from below with respect to the bottom surface of the first jaw 2101. In addition, as an alternative embodiment, the first driving gear 2121′ and the second driving gear 2122′ may be integrally formed with each other or at least coupled to each other. Accordingly, the first driving gear 2121′ and the second driving gear 2122′ may be moved together around the same central shaft 2120AX′.

The first driving gear 2121′ and the second driving gear 2122′ may not be stacked so that each of the first driving gear 2121′ and the second driving gear 2122′ has the same outer surface when disposed. That is, the plurality of teeth and the non-formation area of the first driving gear 2121′ do not overlap the plurality of teeth and the non-formation area of the second driving gear 2122′. For example, at least some of the plurality of teeth of the first driving gear 2121′ may be disposed to overlap the non-formation area of the second driving gear 2122′.

The first driving gear 2121′ and the second driving gear 2122′ may respectively correspond to the first moving part 2111′ and the second moving part 2112′.

When the first moving part 2111′ and the second moving part 2112′ of the reciprocating moving part 2110′ are moved forward and backward, the first driving gear 2121′ and the second driving gear 2122′ of the driving gear 2120′ may be rotated in one direction.

That is, when the first moving part 2111′ is moved in one direction (for example, moved forward), the first driving gear 2121′ may be rotated in one direction by the first rack gear part of the first moving part 2111′, and when the first moving part 2111′ is moved in another direction (for example, moved backward), the first driving gear 2121′ may be rotated in one direction by the second rack gear part of the first moving part 2111′. Accordingly, the operation member 2140 is moved forward during the forward motion and the backward motion of the first moving part 2111′, thereby improving the operation efficiency and the movement of the operation member 2140.

That is, when the second moving part 2112′ is moved in one direction (for example, moved forward), the second driving gear 2122′ may be rotated in one direction by the first rack gear part of the second moving part 2112′, and when the second moving part 2112′ is moved in another direction (for example, moved backward), the second driving gear 2122′ may be rotated in one direction by the second rack gear part of the second moving part 2112′. Accordingly, the operation member 2140 is moved forward during the forward motion and the backward motion of the second moving part 2112′, thereby improving the operation efficiency and the movement of the operation member 2140.

Meanwhile, when the first driving gear 2121′ and the second driving gear 2122′ are disposed, the plurality of teeth and the non-formation area may be disposed to be misaligned with each other. For example, at least some of the plurality of teeth of the first driving gear 2121′ may be disposed to overlap the non-formation area of the second driving gear 2122′.

In this manner, an alternative embodiment does not have the overlapping times, so that the dead zone in which the driving force of the forward and backward motions of the first moving part 2111′ is not transmitted to the first driving gear 2121′ does not temporally coincide with the dead zone in which the driving force of the forward and backward motions of the second moving part 2112′ is not transmitted to the second driving gear 2122′.

For example, at the time when the teeth of the first driving gear 2121′ do not correspond to or engage with the irregularities of the first rack gear part and the irregularities of the second rack gear part in the first moving part 2111′, the teeth of the second driving gear 2122′ may engage with the irregularities of the first rack gear part or the irregularities of the second rack gear part in the second moving part 2112′, and the driving force through the forward and backward motions of the second moving part 2112′ may be transmitted to the second driving gear 2122′.

As a result, when considering the entirety of the driving gear 2120′ and the reciprocating moving part 2110′ on a time basis, the driving force of the reciprocating moving part 2110′ may be transmitted to the driving gear 2120′ without a dead zone, thereby increasing the efficiency of the motion of the transmission gear 2110n′ and the motion of the work member 2140 accordingly.

Since the cartridge and the stapling motion are substantially the same as those described in the embodiments described above, a detailed description thereof is omitted.

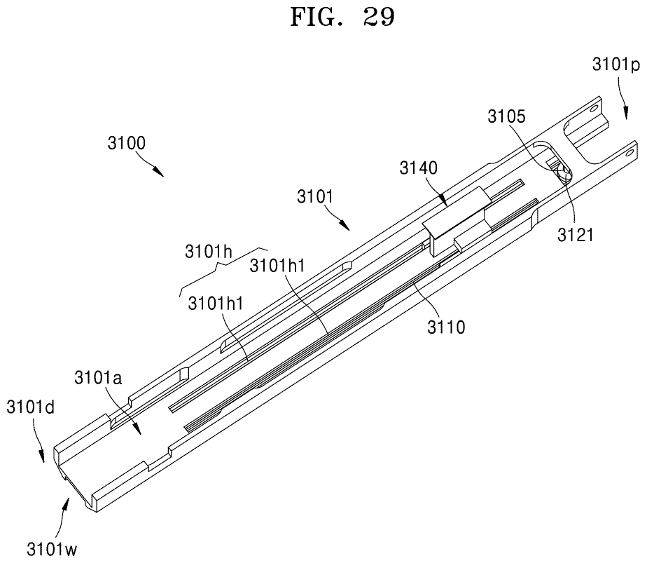

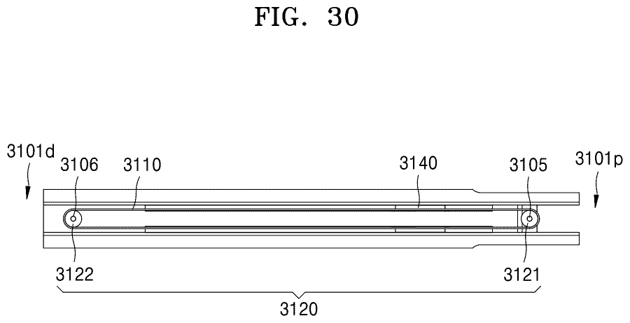

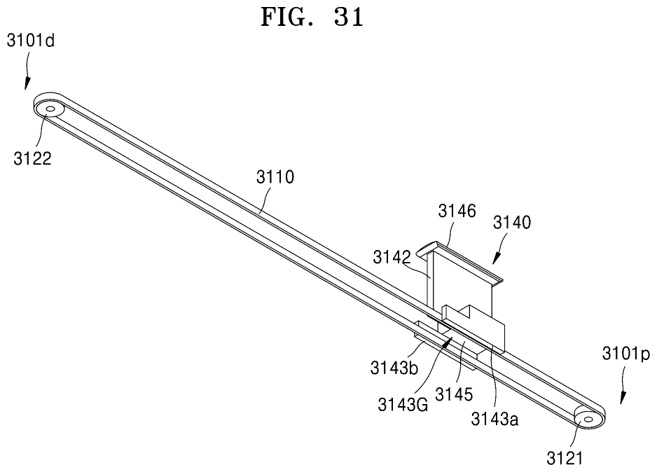

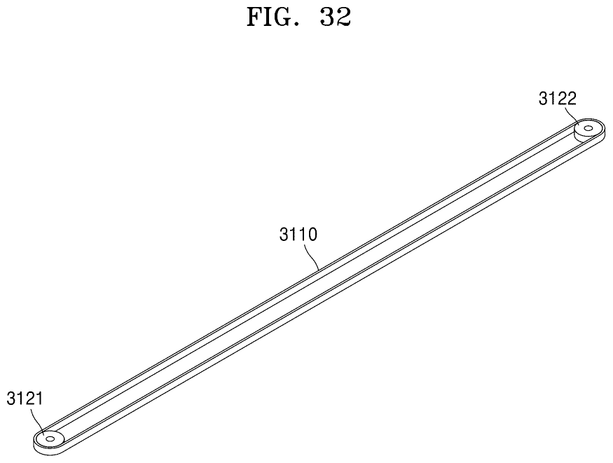

FIG. 29 is a schematic perspective view for describing an end tool of a surgical instrument according to another embodiment. FIG. 30 is a rear view of the end tool of FIG. 29, when viewed from below. FIG. 31 is a perspective view for describing a belt part and a driving module of the end tool of FIG. 29. FIG. 32 is a perspective view schematically illustrating the belt part and the driving module of FIG. 31.

Referring to FIGS. 29 to 32, an end tool 3100 of the present embodiment may include a jaw, a belt part 3110, and a driving module 3120. For convenience of explanation, the description will focus on differences from the embodiments described above. The present embodiment differs from the embodiments described above in terms of the belt part 3110 and the driving module 3120, and thus, the description will focus on such differences.

Referring to FIG. 29, the jaw of the end tool 3100 is illustrated. For convenience of explanation, a first jaw 3101 is mainly illustrated, and a second jaw, a cartridge, and the like are omitted.

The first jaw 3101 may be formed in the shape of an elongated bar as a whole, a cartridge (1500 of FIG. 1) may be accommodated in the first jaw 3101 at a distal end 3101d side, and a rotation shaft may be disposed at a proximal end 3101p so that the first jaw 3101 is rotatable around the rotation shaft. The first jaw 3101 may be formed entirely in the form of a hollow box, of which one surface (upper surface) is removed, so that a cartridge accommodation part 3101a capable of accommodating the cartridge may be formed inside the first jaw 3101. That is, the first jaw 3101 may be formed in an approximately “U” shape in cross section.

One or more first guide grooves 3101h may be formed on the bottom surface of the first jaw 3101, that is, on the bottom surface facing the upper open region in which one surface is removed. The first guide groove 3101h may have a plurality of grooves. For example, the first guide groove 3101h may include a first side groove 3101h1 and a second side groove 3101h2, which are spaced apart from each other. Since this is substantially the same as the first jaw 1101 of the embodiment described above, a detailed description thereof is omitted.

The first jaw 3101 may include an extension groove 3101w in a region facing the back surface thereof. Since this is substantially the same as the first jaw 3101 of the embodiment described above, a detailed description thereof is omitted. However, the inner surface of the extension groove 3101w of the present embodiment does not need to have the same configuration as the plurality of rotation shafts 1101c of the extension groove 1101w of the embodiment described above.

The operation member 3140 may include a main body 3145, a blade 3142, and a side portion 3143. Meanwhile, the operation member 3140 may be used together with the wedge WDG (see FIGS. 7, 17, and 18).

As an alternative embodiment, a clamp 3146 may be further formed in one region of the blade 3142. The side portion 3143 may be formed to have a height in a region of the main body 3145 in a direction opposite to the protruding direction of the blade 3142, and may include a first side portion 3143a and a second side portion 3143b that protrude from both sides in the width direction of the main body 3145. The first side portion 3143a and the second side portion 3143b may be spaced apart from each other, and a separation space 3143G may be formed between the first side portion 3143a and the second side portion 3143b at a location including a region in which the center of the main body 3145 overlaps the blade 3142.

The belt part 3110 and the driving module 3120 may be disposed to move the operation member 3140.

The belt part 3110 may extend to have a length along the longitudinal direction of the first jaw 3101. For example, the belt part 3110 may be formed to be connected to the driving module 3120.

The driving module 3120 may be disposed to drive the belt part 3110 and may include at least a first driving pulley 3121 and a second driving pulley 3122.

The first driving pulley 3121 and the second drive pulley 3122 may be respectively disposed in the direction facing the proximal end 3101p of the first jaw 3101 and the direction facing the distal end 3101d of the first jaw 3101.

For example, the first driving pulley 3121 may be disposed closer to the proximal end 3101p than the operation member 3140, and the second driving pulley 3122 may be disposed closer to the distal end 3101d than the operation member 3140. As a specific example, the operation member 3140 may be disposed between the first driving pulley 3121 and the second driving pulley 3122, and the operation member 3140 may be moved between the first driving pulley 3121 and the second driving pulley 3122.

The first driving pulley 3121 and the second driving pulley 3122 may be respectively rotated around the rotation shafts while being fixed to the first jaw 3101.

The first driving pulley 3121 may be rotated around a first rotation shaft 3105, and the second driving pulley 3122 may be rotated around a second rotation shaft 3106.

The second driving pulley 3122 may be disposed to correspond to the extension groove 3101w of the first jaw 3101. At this time, a protrusion part corresponding to the second rotation shaft 3106 may be formed on one surface of the extension groove 3101w.

The first driving pulley 3121 may be disposed on the same plane as the second driving pulley 3122. For example, the first driving pulley 3121 may be disposed on the extension groove 3101w or at a position beyond the extension groove 3101w, and the first driving pulley 3121 is disposed at a position beyond the extension groove 3101w, the first rotation shaft 3105 may extend from the upper surface of the first jaw 3101 or one region of a frame.

The first driving pulley 3121 or the second driving pulley 3122 of the driving module 3120 may receive a driving force to rotate in one direction or another direction and may transmit the driving force to the belt part 3110. As a specific example, the driving force is transmitted only to the first driving pulley 3121 of the driving module 3120, and the second driving pulley 3122 is an auxiliary pulley that may easily form a loop of the belt part 3110.

To this end, the belt part 3110 may correspond to the first driving pulley 3121 and the second driving pulley 3122 of the driving module 3120. For example, the belt part 3110 is one loop and may be wound while corresponding to the first driving pulley 3121 and the second driving pulley 3122 of the driving module 3120.

The belt part 3110 may be disposed to be accommodated in the extension groove 3101w of the first jaw 3101 so as to correspond to the first driving pulley 3121 and the second driving pulley 3122. At least one region of the belt part 3110 may be rotated along the rotation direction of the first driving pulley 3121 and the second driving pulley 3122 while being accommodated in the extension groove 3101w.

At least one region of the belt part 3110 may be connected to the operation member 3140, and the operation member 3140 may also be moved along the motion direction of the belt part 3110.

For example, when the belt part 3110 is wound around the first driving pulley 3121 and the second driving pulley 3122 of the driving module 3120, two regions, that is, a belt region that is wound in and a belt region that is wound out, may be disposed to face each other and have parallel regions along the width direction of the extension groove 3101w. One of these regions may be connected to one region of the operation member 3140.

As a specific example, referring to FIGS. 30 to 32, etc., the belt part 3110 may be wound around the first driving pulley 3121 and the second driving pulley 3122 of the driving module 3120. Referring to FIG. 31, the belt part 3110 may correspond to the separation space 3143G of the operation member 3140. That is, the two parallel regions of the belt part 3110 may face the first side portion 3143a and the second side portion 3143b of the side portion 3143 of the operation member 3140. One of these regions, for example, the region facing the first side portion 3143a, may be connected and coupled to the first side portion 3143a and moved together, and the regions facing the second side portion 3143b may be spaced apart from each other.

Accordingly, when the belt part 3110 is moved through the rotation of the first driving pulley 3121 and the second driving pulley 3122, for example, when the first driving pulley 3121 and the second driving pulley 3122 in FIG. 30 are rotated in counterclockwise direction, the belt part 3110 may also be rotated in a counterclockwise direction. At this time, since one region of the belt part 3110 is connected and coupled to the first side portion 3143a of the side portion 3143 of the operation member 3140 and moved integrally with each other, the operation member 3140 may also be moved in a left direction of FIG. 31, that is, moved forward, together with the belt part 3110.

In addition, on the contrary, when the first driving pulley 3121 and the second driving pulley 3122 of FIG. 31 are rotated in a clockwise direction, the belt part 3110 may also be rotated in a clockwise direction. At this time, since one region of the belt part 3110 is connected and coupled to the first side portion 3143a of the side portion 3143 of the operation member 3140 and moved integrally with each other, the operation member 3140 may also be moved in a right direction of FIG. 31, that is, moved backward, together with the belt part 3110.

When the rotational motions of the first driving pulley 3121 and the second driving pulley 3122 of the driving module 3120 are controlled in the manner described above, the rotational motion of the belt part 3110 may be controlled and the operation member 3140 may also be moved forward or backward as desired according to the rotational motion of the belt part 3110.

As a result, the number of members for the motion of the operation member 3140 is reduced and the space for arrangement of the members is reduced, thereby improving the operation efficiency and the movement of the operation member 3140 and improving the manufacturing and management efficiency of the end tool 3100.

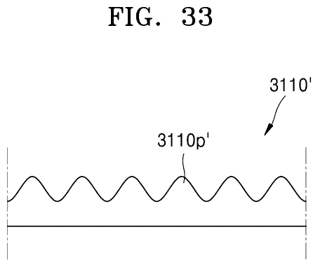

FIG. 33 is a diagram illustrating a modification of the belt part of FIG. 32.

Referring to FIG. 33, a belt part 3110′ may have one or more irregularities 3110p′. For example, the belt part 3110′ may have the form of a timing belt. In addition, the irregularities may be periodic.

In this case, as an alternative embodiment, the first driving pulley (see 3121 of FIG. 32) and the second driving pulley (see 3122 of FIG. 32) may also be formed to have concavo-convex surfaces so as to correspond to the irregularities 3110p′ of the belt part 3110′.

A force of a driving unit may be effectively transmitted to the belt part 3110′ through the structure of the belt part 3110′, and the motion of the belt part 3110′ may be precisely controlled. For example, it is possible to precisely confirm and control the motions of the first driving pulley (see 3121 of FIG. 32) and the second driving pulley (see 3122 of FIG. 32) and the resulting momentum of the belt part 3110′ and it is possible to precisely confirm and control the resulting momentum of the operation member (see 3140 of FIG. 31).

Accordingly, the efficiency and precision of the stapling motion using the operation member may be improved.

Since details of the cartridge and the stapling motion are substantially the same as those described in the embodiment of the end tool 1100, a detailed description thereof is omitted.

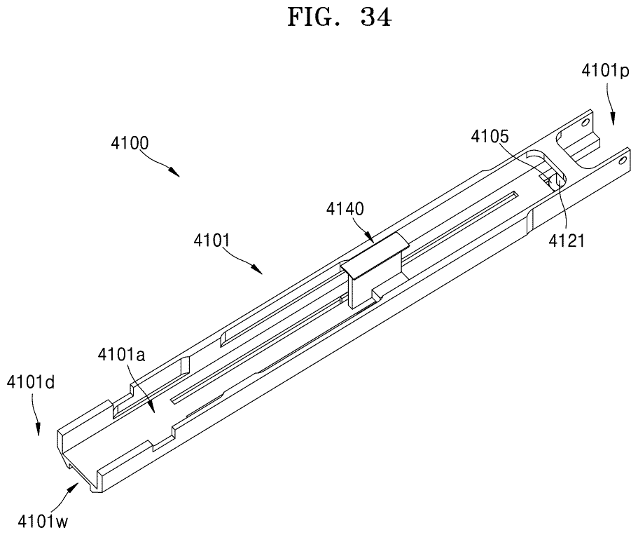

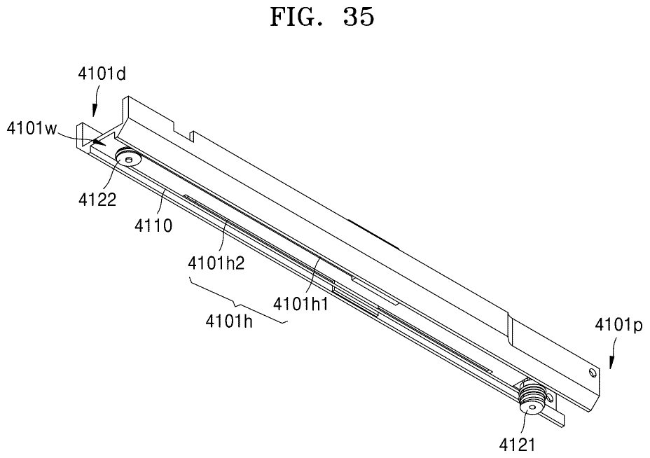

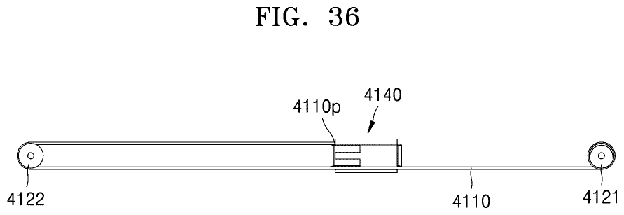

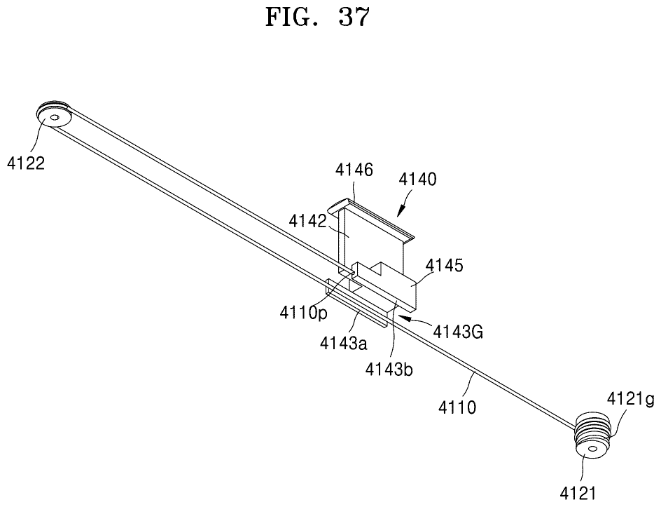

FIG. 34 is a schematic perspective view for describing an end tool of a surgical instrument according to another embodiment. FIG. 35 is a perspective view of the end tool of FIG. 34, when viewed from another direction. FIG. 36 is a plan view for describing a motion wire and a driving module of the end tool of FIG. 34. FIG. 37 is a perspective view for describing the motion wire and the driving module of the end tool of FIG. 34.

Referring to FIGS. 34 to 37, an end tool 4100 of the present embodiment may include a jaw, a motion wire 4110, and a driving unit 4120. For convenience of explanation, the description will focus on differences from the embodiments described above. The present embodiment differs from the embodiments described above in terms of the motion wire 4110 and the driving unit 4120, and thus, the description will focus on such differences.

Referring to FIG. 34, the jaw of the end tool 4100 is illustrated. For convenience of explanation, a first jaw 4101 is mainly illustrated, and a second jaw, a cartridge, and the like are omitted.

The first jaw 4101 may be formed in the shape of an elongated bar as a whole, a cartridge (see, for example, 1500 of FIGS. 1 and 16) may be accommodated in the first jaw 4101 at a distal end 4101d side, and a rotation shaft may be disposed at a proximal end 4101p so that the first jaw 4101 is rotatable around the rotation shaft. The first jaw 4101 may be formed entirely in the form of a hollow box, of which one surface (upper surface) is removed, so that a cartridge accommodation part 4101a capable of accommodating the cartridge may be formed inside the first jaw 4101. That is, the first jaw 4101 may be formed in an approximately “U” shape in cross section.

Referring to FIG. 35, one or more first guide grooves 4101h may be formed on the bottom surface of the first jaw 4101, that is, on the bottom surface facing the upper open region in which one surface is removed. The first guide groove 4101h may have a plurality of grooves. For example, the first guide groove 4101h may include a first side groove 4101h1 and a second side groove 4101h2, which are spaced apart from each other. Since this is substantially the same as the first jaw 1101 of the embodiment described above, a detailed description thereof is omitted.

The first jaw 4101 may include an extension groove 4101w in a region facing the back surface thereof. Since this is substantially the same as the first jaw 4101 of the embodiment described above, a detailed description thereof is omitted. However, the inner surface of the extension groove 4101w of the present embodiment does not need to have the same configuration as the plurality of rotation shafts 1101c of the extension groove 1101w of the embodiment described above.

The operation member 4140 may include a main body 4145, a blade 4142, and a side portion 4143. Meanwhile, the operation member 4140 may be used together with the wedge WDG (see FIGS. 7, 17, and 18).

As an alternative embodiment, a clamp 4146 may be further formed in one region of the blade 4142. The side portion 4143 may be formed to have a height in a region of the main body 4145 in a direction opposite to the protruding direction of the blade 4142, and may include a first side portion 4143a and a second side portion 4143b that protrude from both sides in the width direction of the main body 4145. The first side portion 4143a and the second side portion 4143b may be spaced apart from each other, and a separation space 4143G may be formed between the first side portion 4143a and the second side portion 4143b at a location including a region that overlaps the center of the main body 4145 and the blade 4142.

The motion wire 4110 and the driving unit 4120 may be disposed to move the operation member 4140.

The motion wire 4110 may extend to have a length along the longitudinal direction of the first jaw 4101. For example, the motion wire 4110 may be formed to be connected to the driving unit 4120.

The driving unit 4120 may be disposed to drive the motion wire 4110 and may include at least a driving fixing unit 4121 and a support unit 4122.

The driving fixing unit 4121 and the support unit 4122 may be respectively disposed in the direction facing the proximal end 4101p of the first jaw 4101 and the direction facing the distal end 4101d of the first jaw 4101.

For example, the driving fixing unit 4121 may be disposed closer to the proximal end 4101p than the operation member 4140, and the support unit 4122 may be disposed closer to the distal end 4101d than the operation member 4140. As a specific example, the operation member 4140 may be disposed between the drive fixing unit 4121 and the support unit 4122, and the operation member 4140 may be moved between the driving fixing unit 4121 and the support unit 4122.

Each of the driving fixing unit 4121 and the support unit 4122 may be fixed to the first jaw 4101. Specifically, the driving fixing unit 4121 may be rotated around the rotation shaft while being fixed to the first jaw 4101. Due to the rotational motion, a driving force may be transmitted to the motion wire 4110, and thus, the motion wire 4110 may be moved.

To this end, one region of the motion wire 4110, for example, one end region of the motion wire 4110 may be fixed to the driving fixing unit 4121. One region of the motion wire 4110, for example, one end region of the motion wire 4110 may be wound at least once after being fixed to the driving fixing unit 4121. As a specific example, as illustrated in FIG. 36, one region of the motion wire 4110 may be wound multiple times when the drum-shaped driving fixing unit 4121 is rotated. As an alternative embodiment, one or more grooves 4121g may be formed to effectively wind the motion wire 4110. In addition, as a specific example, the groove 4121g may be formed in the form in which the motion wire 4110 is spirally wound.

Meanwhile, as an alternative embodiment, when the groove 4121g is spirally formed to wind the motion wire 4110 multiple times, the motion wire 4110 may have a length in a winding direction. The length may be equal to or greater than the maximum forward motion distance over which the operation member 4140 is movable forward. Accordingly, the smooth motion of the motion wire 4110 necessary for the forward motion of the operation member 4140 and the resulting winding region may be ensured, and the motion wire 4110 may be wound in close contact with the groove 4121g, thereby improving driving efficiency.

In addition, as another alternative embodiment (not shown), the driving fixing unit 4121 may have the form of a disk-shaped pulley instead of a drum-shaped pulley, and the motion wire 4110 may be wound around one groove multiple times.

The support unit 4122 may be disposed to correspond to the extension groove 4101w of the first jaw 4101. The motion wire 4110 may be wound around the support unit 4122. The motion wire 4110, which is fixed to the driving fixing unit 4121 and pulled according to the rotation of the driving fixing unit 4121, may be moved while guiding the path according to the support unit 4122. To this end, the support unit 4122 may have a groove corresponding to the motion wire 4110 on the outer surface thereof and may have an outer circumferential surface in a circular or arc shape. Specifically, the support unit 4122 may have the form of a pulley. As an alternative embodiment, the support unit 4122 may be formed to be moved around one shaft together with the motion wire 4110.

The driving fixing unit 4121 of the driving unit 4120 may receive a driving force so as to be rotated and may transmit the driving force to the motion wire 4110.

To this end, one region of the motion wire 4110, for example, one end portion of the motion wire 4110 is fixed to the driving fixing unit 4121 of the driving unit 4120, and the other end portion (4110p of FIG. 37) thereof extends toward the support unit 4122 and is supported by the support unit 4122. After one region thereof is wound and the path is changed to face the driving fixing unit 4121, it may be connected to a region of the operation member 4140, for example, to a region of the second side portion 3143b of the side portion 4143, or as a specific example, a region that does not correspond to the separation space 3143G.

The motion wire 4110 may be disposed to be accommodated in the extension groove 4101w of the first jaw 4101 so as to correspond to the driving fixing unit 4121 and the support unit 4122. At least one region of the motion wire 4110 may be wound and rotated according to the rotation of the driving fixing unit 4121 while being accommodated in the extension groove 4101w.

At least one region of the motion wire 4110 may be connected to the operation member 4140, and the operation member 4140 may also be moved along the motion direction of the motion wire 4110.

A region of the motion wire 4110 facing the support unit 4122 from a portion connected to the driving fixing unit 4121 and a region (4110p of FIG. 37) of the motion wire 4110 connected to the operation member 4140 from a portion wound around the support unit 4122 may be disposed to face each other and have parallel regions along the width direction of the extension groove 4101w.

As a specific example, referring to FIG. 37, a region of the motion wire 4110 facing the support unit 4122 from a portion connected to the driving fixing unit 4121 may correspond to the separation space 4143G of the operation member 4140, may face the first side portion 4143a of the side portion 4143 of the operation member 4140, and may be moved while being spaced apart from the first side portion 4143a.

A region (4110p of FIG. 37) of the motion wire 4110 connected to the operation member 4140 from a portion wound around the support unit 4122 may be connected and fixed to a front side among the regions of the second side portion 4143b of the side portion 4143, that is, one surface facing the support unit 4122 outside the region facing the separation space 4143G.

Accordingly, when the motion wire 4110 is moved through the rotation of the driving fixing unit 4121, for example, when the driving fixing unit 4121 of FIG. 37 is rotated in a clockwise direction, that is, when the driving fixing unit 4121 is moved in the direction of winding the motion wire 4110, the motion wire 4110, one end portion of which is fixed to the driving fixing unit 4121, is moved toward the driving fixing unit 4121 and is wound around the driving fixing unit 4121. In this case, a region of the motion wire 4110 facing the support unit 4122 from a portion connected to the driving fixing unit 4121 may be moved toward the driving fixing unit 4121, and a region (4110p of FIG. 37) of the motion wire 4110 connected to the operation member 4140 from a portion wound around the support unit 4122 may be moved toward the support unit 4122. As a result, the operation member 4140 connected to the end region (4110p of FIG. 37) of the motion wire 4110 may also be moved toward the support unit 4122, that is, the operation member 4140 may be moved forward in the distal end direction.

By controlling the rotational motion of the driving fixing unit 4121 of the driving unit 4120 as described above, the rotational motion of the motion wire 4110 may be controlled and the operation member 4140 may be moved according to the rotational motion of the motion wire 4110.

As a result, the number of members for the motion of the operation member 4140 is reduced and the space for arrangement of the members is reduced, thereby improving the operation efficiency and the movement of the operation member 4140 and improving the manufacturing and management efficiency of the end tool 4100.

Since details of the cartridge and the stapling motion are substantially the same as those described in the embodiment of the end tool 1100, a detailed description thereof is omitted.

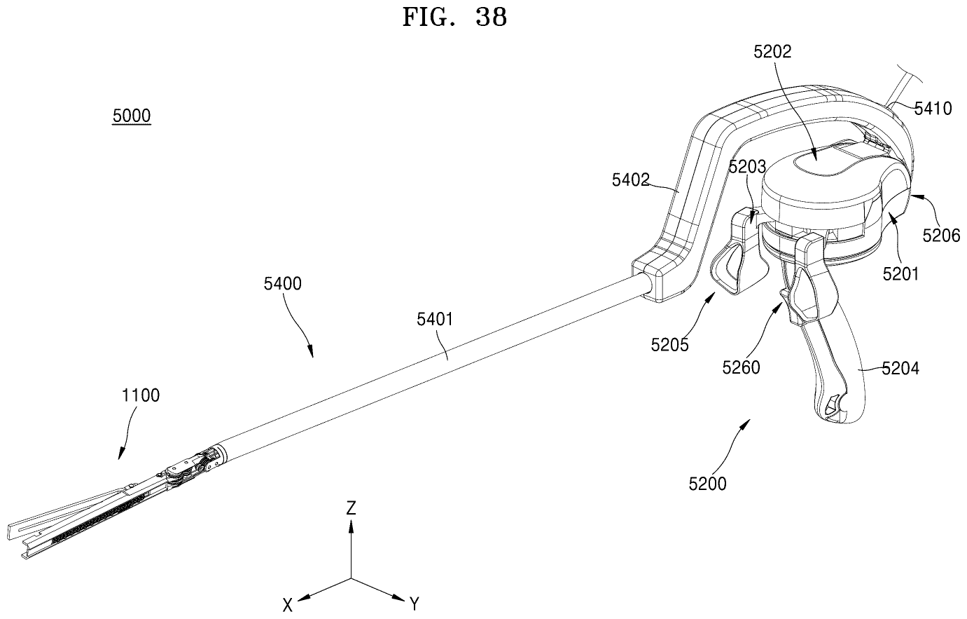

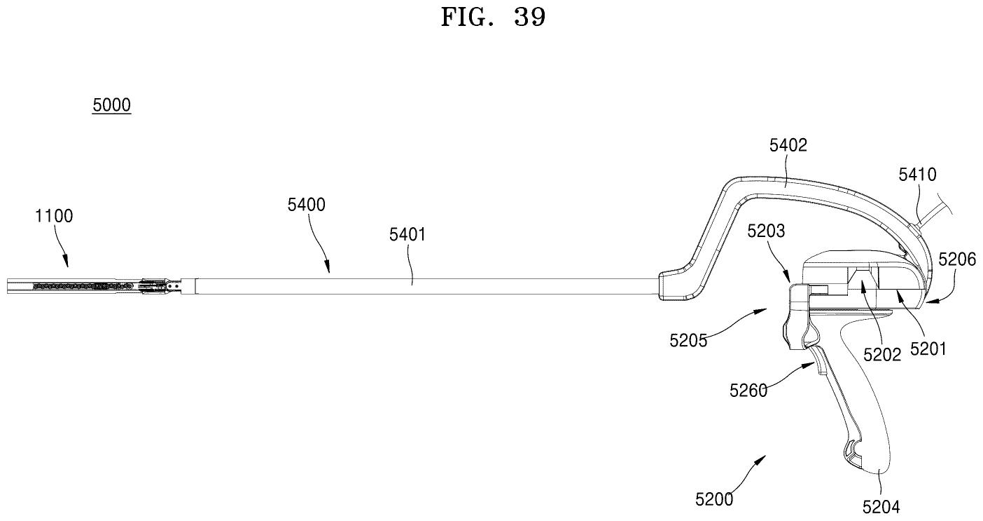

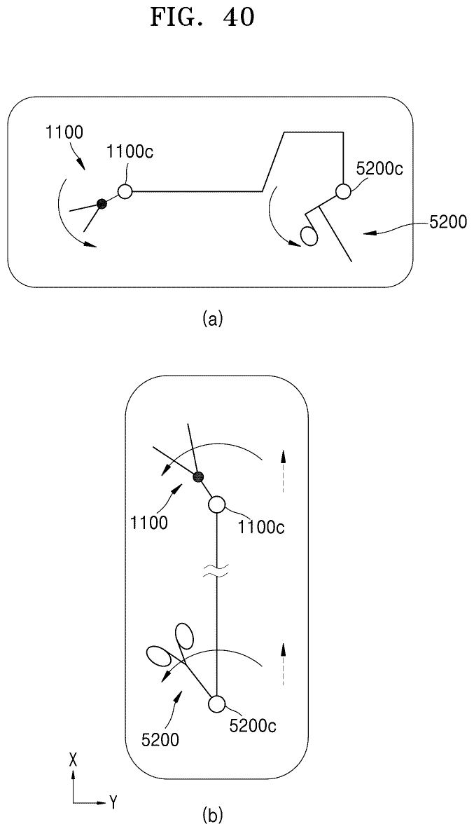

FIG. 38 is a schematic perspective view of a surgical instrument according to another embodiment, FIG. 39 is a side view of the surgical instrument of FIG. 38, and FIG. 40 is a diagram for schematically describing the operation concept of the surgical instrument of FIG. 38.

Referring to FIGS. 38 and 39, a surgical instrument 5000 according to the present embodiment may include an end tool 1100, a manipulation part 5200, a power transmission part (not shown), and a connection part 5400.

Since the end tool 1100 may include the end tool 1100 of the embodiment illustrated in FIG. 1 as it is, a detailed description thereof is omitted. In addition, although not shown, as an alternative embodiment, it is obvious that the surgical instrument 5000 may selectively apply one of the end tools 2100, 3100, and 4100 described above. The modifications described above may be selectively applied, and the end tool 1100 is shown only for convenience of explanation.

The connection part 5400 may be formed in the shape of a hollow shaft, and one or more wires and electric wires may be accommodated therein. The manipulation part 5200 may be coupled to one end portion of the connection part 5400, and the end tool 1100 may be coupled to the other end portion of the connection part 5400, so that the connection part 5400 may serve to connect the manipulation part 5200 to the end tool 1100.

As an alternative embodiment, the connection part 5400 may include a straight portion 5401 and a bent portion 5402. The straight portion 5401 may be formed on the side to be coupled to the end tool 1100, and the bent portion 5402 may be formed on the side to be coupled to the manipulation part 5200. As described above, since the end portion of the connection part 5400 on the manipulation part 5200 side is bent, a pitch manipulation part 5201, a yaw manipulation part 5202, and an actuation manipulation part 5203 are formed on an extension line of the end tool 1100 or is formed adjacent to the extension line. In another aspect, it may be said that at least a portion of the pitch manipulation part 5201 and the yaw manipulation part 5202 is accommodated in a concave portion formed by the bent portion 5402. Due to the shape of the bent portion 5402, the shapes and operations of the manipulation part 5200 and the end tool 1100 may more intuitively coincide with each other.

Meanwhile, a plane on which the bent portion 5402 is formed may be a pitch plane, that is, a plane substantially the same as an XZ plane of FIG. 38. As such, interference between the manipulation parts may be reduced by forming the bent portion 5402 on substantially the same plane as the XZ plane. For intuitive motions of the end tool and the manipulation part, other configurations other than the XZ plane may also be possible.

Meanwhile, as an alternative embodiment, a connector 5410 may be formed in the bent portion 5402. The connector 5410 may be connected to an external power source (not shown) and the connector 5410 may also be connected to the end tool 1100 through an electric wire, so that electrical energy supplied from the external power source (not shown) may be transmitted to the end tool 1100.

The manipulation part 5200 is formed at one end portion of the connection part 5400 and provided as an interface to be directly controlled by a medical doctor, for example, a handle 5204, a tongs shape, a stick shape, a lever shape, or the like, and when the medical doctor controls the manipulation part 5200, the end tool 1100, which is connected to the interface and inserted into the body of a surgical patient, performs a certain motion, thereby performing surgery. Although it is illustrated that the manipulation part 5200 is formed in the shape of a handle that is rotatable with a finger inserted thereinto, the concept of the disclosure is not limited thereto, and various types of manipulation parts that may be connected to the end tool 1100 and manipulate the end tool 1100 are possible. In addition, the manipulation part 5200 may be variously controlled through one or more driving shafts disposed in the direction from a distal end 5205 to a proximal end 5206 of the manipulation part 5200.

In addition, the manipulation part 5200 of the surgical instrument 5000 may further include a staple manipulation part 5260 that performs stapling and cutting of the end tool 1100.

Meanwhile, the present embodiment may also be applied to a robot-driven type instead of a manual type that is directly controlled by a person. In this case, the end tool 1100 may be connected directly to a robot, instead of the manual-type manipulation part 5200, or connected to a robot driving arm or other areas through the connection part 5400.

The end tool 1100 of the surgical instrument 5000 according to the present embodiment may be configured to be rotatable in at least one direction. For example, the end tool 1100 of the surgical instrument 5000 according to the present embodiment may be configured to perform a pitch motion around the Y-axis of FIG. 38 and perform a yaw motion and an actuation motion around the Z-axis.

The pitch, yaw, and actuation motions used in the disclosure are defined as follows.

First, the pitch motion refers to a motion in which the end tool 1100 is rotated in a vertical direction with respect to the extension direction (X-axis direction of FIG. 38) of the connection part 5400, that is, a motion in which the end tool 1100 is rotated around the Y-axis of FIG. 38. In other words, the pitch motion refers to a motion in which the end tool 1100 extending from the connection part 5400 in the extension direction (X-axis direction of FIG. 38) of the connection part 5400 is rotated up and down around the Y-axis with respect to the connection part 5400.

Next, the yaw motion refers to a motion in which the end tool 1100 is rotated in a left-and-right direction with respect to the extension direction (X-axis direction of FIG. 38) of the connection part 5400, that is, a motion in which the end tool 1100 is rotated around the Z-axis of FIG. 38. In other words, the yaw motion refers to a motion in which the end tool 1100 extending from the connection part 5400 in the extension direction (X-axis direction of FIG. 38) of the connection part 5400 is rotated left and right around the Z-axis with respect to the connection part 5400. That is, the yaw motion refers to a motion in which two jaws 1001 and 1002 formed in the end tool 1100 are rotated around the Z-axis in the same direction.

Meanwhile, the actuation motion refers to a motion in which the end tool 1100 is rotated around the same rotation shaft as the yaw motion and the two jaws 1001 and 1002 are opened or closed while rotating in opposite directions. That is, the actuation motion refers to a motion in which the two jaws 1001 and 1002 formed in the end tool 1100 are rotated around the Z-axis in opposite directions.

The power transmission part (not shown) serves to transmit the driving force of the manipulation part 5200 to the end tool 1100 by connecting the manipulation part 5200 to the end tool 1100. The power transmission part may include a plurality of wires, pulleys, links, nodes, gears, and the like.

FIG. 40 may a diagram describing an example of the operation of the surgical instrument 5000 of FIG. 38.

Specifically, referring to FIGS. 40A and 40B, in the surgical instrument, the end tool 1100 is formed in front of a rotation center 1100c of the end tool 1100, and the manipulation part 5200 is also formed in front of a rotation center 5200c of the manipulation part 5200, so that the operations of the manipulation part 5200 and the end tool 1100 intuitively coincide with each other. Expressing these features differently, the surgical instrument according to an embodiment is formed so that at least a portion of the manipulation part is closer to the end tool (than its own joint) with respect to its own joint at one or more moments during the manipulation process.

In the surgical instrument according to an embodiment, since both the end tool and the manipulation part are moved with respect to the rotation center formed at the rear, it may be said that the operations may intuitively coincide with each other in terms of structure. It may also be said that just as the moving part of the end tool is moved with respect to the rotation center formed at the rear, the moving part of the manipulation part is also moved with respect to the corresponding rotation center formed at the rear, and thus, the operations intuitively coincide with each other in terms of structure. This allows users to quickly and intuitively control the direction of the end tool and reduces the possibility of making mistakes.

As an alternative embodiment, the surgical instrument to which the end tool of the present embodiment is applied may be driven in various ways. For example, the surgical instrument to which the end tool of the present embodiment is applied may also be applied to embodiments in which the central shafts of the end tool are changed front and rear, or the manipulation part and the central shaft thereof are changed front and rear.

The disclosure has been described with reference to the embodiments illustrated in the drawings, but this is only an example. It will be understood by those of ordinary skill in the art that various modifications and equivalents may be made thereto. Accordingly, the true technical protection scope of the disclosure should be defined by the technical spirit of the appended claims.

The embodiments may be represented by functional block configurations and various processing steps. These functional blocks may be implemented in various numbers of hardware and/or software configurations that execute specific functions. For example, the embodiments may directly employ integrated circuit configurations, such as memory, processing, logic, look-up tables, and the like, which may execute various functions under the control of one or more microprocessors or other control devices.

Specific executions described in the embodiments are one embodiment, which does not limit the scope of the embodiments in any way. For the sake of conciseness of the specification, descriptions of conventional electronic components, control systems, software, and other functional aspects of the methods may be omitted. In addition, connecting lines or connecting members illustrated in the drawings are intended to represent exemplary functional connections and/or physical or circuit connections. In an actual device, it may appear as a variety of alternative or additional functional, physical, or circuit connections. In addition, when there is no specific mention such as “essential,” “important,” etc., it may not be a necessary component for the application of the disclosure.

The use of the term “the” and similar demonstratives in the specification of the embodiments (in particular, the claims) is to be construed to cover both the singular and the plural. In addition, when a range is described in the embodiments, it includes the invention to which individual values within the range are applied (unless otherwise indicated herein). This is the same as stating each individual value constituting the above range in the detailed description. Finally, operations constituting methods according to embodiments may be performed in any suitable order unless otherwise indicated herein or otherwise clearly contradicted by context. The embodiments are not necessarily limited by the order of description of operations. The use of any and all examples or exemplary terms (e.g., “such as”) provided in the embodiments is simply intended to describe the embodiments in detail, and the scope of the embodiments is not limited by the examples or exemplary terms unless otherwise claimed. In addition, it will be understood by those of ordinary skill in the art that various modifications, combinations and changes may be made according to design conditions and factors within the scope of the appended claims or equivalents thereof.

The end tool and the surgical instrument according to the disclosure may precisely perform the movement of the operation member when performing one or more operations included in laparoscopic surgery or various other surgeries.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An end tool comprising:
a jaw configured to accommodate at least one region of an operation member that is movable in at least one direction;
a reciprocating moving part spaced apart from the operation member, disposed on a proximal end side of the jaw rather than the operation member, and configured to receive a driving force from a separate driving part and perform at least a linear reciprocating motion; and
a plurality of transmission gears configured to receive a driving force of a linear motion of the reciprocating moving part, perform a rotational motion, and transmit the driving force to the operation member so that the operation member is moved in the one direction.

2. The end tool of claim 1, further comprising at least one driving gear comprising a plurality of teeth and configured to come into contact with a region of the reciprocating moving part, perform a rotational motion by the reciprocating moving part, and transmit the rotational motion to one transmission gear among the plurality of transmission gears.

3. The end tool of claim 2, wherein the reciprocating moving part comprises a main body and at least one contact part extending from the main body in a direction toward the operation member, and
the at least one contact part is configured to come into contact with the at least one driving gear and transmit a force so that the driving gear is rotated.

4. The end tool of claim 3, wherein, when the at least one driving gear is rotated, a rotational force is transmitted to the one transmission gear connected to the at least one driving gear and the plurality of transmission gears sequentially disposed, so that the plurality of transmission gears are rotated.

5. The end tool of claim 3, wherein the at least one contact part of the reciprocating moving part comprises a first contact part and a second contact part that extend from the main body, are spaced apart from each other, and have different lengths.

6. The end tool of claim 5, wherein the at least one driving gear comprises a first driving gear and a second driving gear respectively corresponding to the first contact part and the second contact part.

7. The end tool of claim 1, wherein a rack member is disposed in at least one region of the jaw,
the rack member is configured to engage with the plurality of transmission gears,
the rack member is configured to be moved forward and backward by a driving force of the plurality of transmission gears, and
the operation member is configured to be moved by the rack member.

8. The end tool of claim 7, wherein the operation member is configured to be moved integrally with the rack member.

9. The end tool of claim 8, wherein the operation member comprises a main body, a blade, and a side portion connected to the main body and formed on a side of the main body opposite to a side facing the blade,
the rack member is disposed between a first side portion and a second side portion of the side portion, which are spaced apart from each other, and
the rack member comprises a first rack member corresponding to the first side portion and a second rack member corresponding to the second side portion.

10. The end tool of claim 9, wherein the plurality of transmission gears comprise a plurality of first transmission gears and a plurality of second transmission gears disposed to be sequentially misaligned with each other,
the plurality of first transmission gears are engaged with the first rack member, and
the plurality of second transmission gears are engaged with the second rack member.

11. The end tool of claim 7, wherein the jaw comprises a first jaw and a second jaw facing each other and configured to perform an open motion and a close motion,
the first jaw comprises an accommodation part configured to accommodate a plurality of staples, and
the rack member is disposed in a region opposite to a surface of the first jaw on which the accommodation part is formed.

12. The end tool of claim 11, wherein the plurality of transmission gears are disposed in the region opposite to the surface of the first jaw on which the accommodation part is formed, so as to correspond to the rack member.

13. The end tool of claim 12, wherein the first jaw comprises an extension groove formed along a longitudinal direction of the first jaw on a surface opposite to the surface of the first jaw on which the accommodation part is formed, and
the rack member and the plurality of transmission gears are disposed in the extension groove.

14. The end tool of claim 7, wherein the rack member is formed separately from the operation member, is disposed closer to a proximal end of the jaw than the operation member, and pushes the operation member during the motion of the rack member so that the operation member is moved.

15. The end tool of claim 1, further comprising a driving gear comprising a concavo-convex portion having a plurality of teeth on an outer circumferential surface thereof and a non-formation area which is adjacent to the concavo-convex portion and in which teeth are not formed, the driving gear being in contact with one region of the reciprocating moving part and being configured to perform a rotation motion by the reciprocating moving part and transmit the rotation motion to one transmission gear among the plurality of transmission gears.

16. The end tool of claim 15, wherein the reciprocating moving part comprises a first rack gear part and a second rack gear part facing each other, and a main body connecting the first rack gear part to the second rack gear part, and
the first rack gear part and the second rack gear part are formed to have concavo-convex portions corresponding to the plurality of teeth of the driving gear.

17. The end tool of claim 16, wherein, when the reciprocating moving part is moved forward or backward, the first rack gear part or the second rack gear part are engaged with the plurality of teeth of the driving gear so that the driving gear is rotated, and
a rotational force of the driving gear is transmitted to one transmission gear connected to the driving gear among the plurality of transmission gears and the plurality of transmission gears sequentially disposed, so that the plurality of transmission gears are rotated.

18. The end tool of claim 17, wherein, when the reciprocating moving part starts a forward motion and repeats forward and backward motions in a state in which the second rack gear part of the reciprocating moving part is engaged with the plurality of teeth of the driving gear, the driving gear is rotated in one rotation direction and the operation member is moved forward.

19. The end tool of claim 17, wherein, when the reciprocating moving part starts a forward motion and repeats forward and backward motions in a state in which the first rack gear part of the reciprocating moving part is engaged with the plurality of teeth of the driving gear, the driving gear is rotated in one rotation direction and the operation member is moved backward.

20. The end tool of claim 17, wherein the reciprocating moving part comprises a first reciprocating moving part and a second reciprocating moving part that are disposed up and down and moved forward or backward.
